(19) United States
(12) Patent Application Publication
(10) Pub. No.: US 2022/0362304 A1
(43) Pub. Date: Nov. 17, 2022

(54) ADOPTIVE CELL THERAPY

(71) Applicant: 2seventy bio, Inc., Cambridge, MA (US)

(72) Inventor: JAMES BRIAN ROTTMAN, SUDBURY, MA (US)

(73) Assignee: 2seventy bio, Inc., Cambridge, MA (US)

(21) Appl. No.: 17/440,372

(22) PCT Filed: Mar. 20, 2020
(86) PCT No.: PCT/US2020/024016

§ 371 Date: Sep. 17, 2021

Related U.S. Application Data
(60) Provisional application No. 62/821,419, filed on Mar. 20, 2019.

(51) Int. Cl. A61K 35/28; A61K 35/17; A61K 38/17; C07K 16/28; A61P 35/00

(52) U.S. Cl. CPC A61K 35/28; A61K 35/17; A61K 38/1774; C07K 16/2803; A61P 35/00; C07K 2317/622; C07K 2317/569; C07K 2319/33

(57) ABSTRACT

The invention provides improved methods for adoptive cell therapies for the treatment of cancer.

### RELATED APPLICATIONS

This application claims the benefit of U.S. Provisional Application No. 62/821,419, filed Mar. 20, 2019. The entire teachings of the above application are incorporated herein by reference.

### BACKGROUND

### Technical Field

The present invention relates to improved adoptive cell therapy compositions and related methods. More particularly, the invention relates to adoptive cell therapies that improve both acute and long-term treatment of immune system disorders.

### Description of the Related Art

Cancer is a significant health problem throughout the world. Based on rates from the International Agency for Research on Cancer (IARC), in 2012 there were 14.1 million new cancer cases and 8.2 million cancer deaths worldwide. In 2015, cancer was the second leading cause of death globally, and was responsible for 8.8 million deaths; nearly 1 in 6 deaths were due to cancer. By 2030, the global burden is expected to grow to 21.7 million new cancer cases and 13 million cancer deaths simply due to the growth and aging of the population. The future burden will probably be even larger because of the adoption of western lifestyles, such as smoking, poor diet, physical inactivity, and fewer childbirths, in economically developing countries. The total annual economic cost of cancer in 2010 was estimated at approximately US$1.16 trillion. The economic impact of cancer is significant and is increasing.

Although advances have been made in detection, prevention, and treatment of cancer, a universally successful therapeutic strategy has yet to be realized. The response to various forms of cancer treatment is mixed. Traditional methods of treating cancers, including chemotherapy and radiotherapy, have limited utility due to toxic side effects. Immunotherapies with therapeutic antibodies have also provided limited success, due in part to poor pharmacokinetic profiles, rapid elimination of antibodies by serum proteases and filtration at the glomerulus, and limited penetration into the tumor site and expression levels of the target antigen on tumor cells. Attempts to use genetically modified cells expressing chimeric antigen receptors (CARs) have also met with limited success. The effects of these cells are often short-lived due to poor in vivo expansion of CAR T cells, rapid disappearance of the cells after infusion, disappointing clinical activity, and antigen escape. In some instances, initial tumor burden is reduced and decreases CAR T cell persistence, which in turn, could lead to tumor outgrowth and leave patients more vulnerable to relapse.

### BRIEF SUMMARY

The invention generally provides improved adoptive cell therapies and methods of making and using the same. More particularly, the invention provides methods using adoptive cell therapies to provide both short-term and long-term treatment, prevention, and/or amelioration of immune system disorders.

In various embodiments, a method of treating a cancer in a subject is provided comprising administering to the subject, an effective amount of human CD34⁺ hematopoietic stem and progenitor cells transduced with a lentiviral vector encoding a first engineered antigen receptor, wherein the first engineered antigen receptor comprises a binding domain that binds one or more target antigens present on a cancer cell; and administering to the subject, an effective amount of human immune effector cells transduced with the lentiviral vector encoding a second engineered antigen receptor; thereby treating the cancer in the subject.

In particular embodiments, the subject undergoes a myeloablative conditioning regimen, reduced intensity conditioning regimen, or nonmyeloablative conditioning regimen, before administering the CD34⁺ HSPCs and immune effector cells.

In certain embodiments, the human CD34⁺ hematopoietic stem and progenitor cells are allogenic to the subject.

In some embodiments, the human CD34⁺ hematopoietic stem and progenitor cells are autologous to the subject.

In particular embodiments, the human immune effector cells are allogenic to the subject.

In further embodiments, the human immune effector cells are autologous to the subject.

In certain embodiments, the human immune effector cells comprise T cells.

In additional embodiments, the human immune effector cells comprise T cells that express CD3⁺, CD4⁺, CD8⁺, or a combination thereof.

In particular embodiments, the human immune effector cells comprise cytotoxic T lymphocytes (CTLs), tumor infiltrating lymphocytes (TILs), and/or helper T cells.

In certain embodiments, the human immune effector cells comprise natural killer (NK) cells or natural killer T (NKT) cells.

In some embodiments, the cancer is a solid cancer.

In particular embodiments, the cancer is a solid cancer selected from the group consisting of: adrenal cancer, adrenocortical carcinoma, anal cancer, appendix cancer, astrocytoma, atypical teratoid/rhabdoid tumor, basal cell carcinoma, bile duct cancer, bladder cancer, bone cancer, brain/CNS cancer, breast cancer, bronchial tumors, cardiac tumors, cervical cancer, cholangiocarcinoma, chondrosarcoma, chordoma, colon cancer, colorectal cancer, craniopharyngioma, ductal carcinoma in situ (DCIS) endometrial cancer, ependymoma, esophageal cancer, esthesioneuroblastoma, Ewing's sarcoma, extracranial germ cell tumor, extragonadal germ cell tumor, eye cancer, fallopian tube cancer, fibrous histiosarcoma, fibrosarcoma, gallbladder cancer, gastric cancer, gastrointestinal carcinoid tumors, gastrointestinal stromal tumor (GIST), germ cell tumors, glioma, glioblastoma, head and neck cancer, hemangioblastoma, hepatocellular cancer, hypopharyngeal cancer, intraocular melanoma, kaposi sarcoma, kidney cancer, laryngeal cancer, leiomyosarcoma, lip cancer, liposarcoma, liver cancer, lung cancer, non-small cell lung cancer, lung carcinoid tumor, malignant mesothelioma, medullary carcinoma, medulloblastoma, menangioma, melanoma, Merkel cell carcinoma, midline tract carcinoma, mouth cancer, myxosarcoma, myelodysplastic syndrome, myeloproliferative neoplasms, nasal cavity and paranasal sinus cancer, nasopharyngeal cancer, neuroblastoma, oligodendroglioma, oral cancer, oral cavity cancer, oropharyngeal cancer, osteosarcoma, ovarian cancer, pancreatic cancer, pancreatic islet cell tumors, papillary carcinoma, paraganglioma, parathyroid cancer, penile cancer, pharyngeal cancer, pheochromocytoma, pinealoma, pituitary tumor, pleuropulmonary blastoma, primary peritoneal cancer, prostate cancer, rectal cancer, retinoblastoma, renal cell carcinoma, renal pelvis and ureter cancer, rhabdomyosarcoma, salivary gland cancer, sebaceous gland carcinoma, skin cancer, soft tissue sarcoma, squamous cell carcinoma, small cell lung cancer, small intestine cancer, stomach cancer, sweat gland carcinoma, synovioma, testicular cancer, throat cancer, thymus cancer, thyroid cancer, urethral cancer, uterine cancer, uterine sarcoma, vaginal cancer, vascular cancer, vulvar cancer, and Wilms Tumor.

In further embodiments, the cancer is a solid cancer selected from the group consisting of: liver cancer, pancreatic cancer, lung cancer, breast cancer, bladder cancer, brain cancer, bone cancer, thyroid cancer, kidney cancer, and skin cancer.

In some embodiments, the cancer is a liquid cancer or hematological cancer.

In particular embodiments, the hematological malignancy is a B cell malignancy.

In particular embodiments, the B cell malignancy is selected from the group consisting of: leukemias, lymphomas, and myelomas.

In additional embodiments, the B cell malignancy is selected from the group consisting of: acute lymphocytic leukemia (ALL), acute myeloid leukemia (AML), myeloblastic, promyelocytic, myelomonocytic, monocytic, erythroleukemia, hairy cell leukemia (HCL), chronic lymphocytic leukemia (CLL), and chronic myeloid leukemia (CML), chronic myelomonocytic leukemia (CMML) and polycythemia vera, Hodgkin lymphoma, nodular lymphocyte-predominant Hodgkin lymphoma, Burkitt lymphoma, small lymphocytic lymphoma (SLL), diffuse large B-cell lymphoma, follicular lymphoma, immunoblastic large cell lymphoma, precursor B-lymphoblastic lymphoma, mantle cell lymphoma, marginal zone lymphoma, mycosis fungoides, anaplastic large cell lymphoma, Sézary syndrome, precursor T-lymphoblastic lymphoma, multiple myeloma, overt multiple myeloma, smoldering multiple myeloma, plasma cell leukemia, non-secretory myeloma, IgD myeloma, osteosclerotic myeloma, solitary plasmacytoma of bone, and extramedullary plasmacytoma.

In certain embodiments, the B cell malignancy is multiple myeloma.

In further embodiments, the one or more target antigens is selected from the group consisting of: tumor associated antigens (TAA), tumor specific antigens (TSA), NKG2D ligands, γδ T cell receptor (TCR) ligands, and αβ TCR ligands.

In particular embodiments, the one or more target antigens is selected from the group consisting of: alpha folate receptor (FRα), αvβ6 integrin, B cell maturation antigen (BCMA), B7-H3 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IL-10Rα, IL-13Rα2, Kappa, cancer/testis antigen 2 (LAGE-1A), Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), trophoblast glycoprotein (TPBG), UL16-binding protein (ULBP) 1, ULBP2, ULBP3, ULBP4, ULBP5, ULBP6, vascular endothelial growth factor receptor 2 (VEGFR2), and Wilms tumor 1 (WT-1).

In some embodiments, the one or more target antigens is selected from the group consisting of: BCMA, CD19, CD20, CD22, CD23, CD33, CD37, CD38, CD52, CD79a, CD79b, CD80, C123, and HLA-DR.

In additional embodiments, the one or more target antigens comprises BCMA.

In some embodiments, the one or more target antigens comprises CD19.

In certain embodiments, the one or more target antigens comprises CD20.

In some embodiments, the one or more target antigens comprises CD22.

In further embodiments, the one or more target antigens comprises CD23.

In particular embodiments, the one or more target antigens comprises CD33.

In certain embodiments, the one or more target antigens comprises CD37.

In particular embodiments, the one or more target antigens comprises CD38.

In additional embodiments, the one or more target antigens comprises CD79a.

In particular embodiments, the one or more target antigens comprises CD79b.

In certain embodiments, the one or more target antigens comprises CD123.

In some embodiments, the cancer expresses a first target antigen and a second target antigen.

In some embodiments, the first and second target antigens are expressed on different cancer cells.

In additional embodiments, the first and second target antigens are expressed on the same cancer cells.

In further embodiments, the first and second engineered antigen receptors are selected from the group consisting of: a chimeric antigen receptor (CAR), an αβ T cell receptor (αβ-TCR), a γδ T cell receptor (γδ-TCR), and a dimerizing agent regulated immunoreceptor complex (DARIC).

In additional embodiments, the first and second engineered antigen receptors are the same.

In particular embodiments, the first and second engineered antigen receptors are a CAR.

In particular embodiments, the CAR comprises: one or more target antigen binding domains; a transmembrane domain; one or more intracellular costimulatory signaling domains; and/or a primary signaling domain.

In certain embodiments, the one or more target antigen binding domains is selected from the group consisting of: a Camel Ig, a Llama Ig, an Alpaca Ig, Ig NAR, a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), and a single-domain antibody (sdAb, a camelid VHH, Nanobody).

In some embodiments, the one or more target antigen binding domains comprises one or more scFvs.

In further embodiments, the one or more target antigen binding domains comprises one or more VHHs.

In particular embodiments, the CAR comprises: an scFv; a CD28 transmembrane domain or a CD8α transmembrane domain; a 4-1BB, OX-40, or CD28 costimulatory domain; and a CD3ζ primary signaling domain.

In certain embodiments, the CAR comprises: a VHH; a CD28 transmembrane domain or a CD8α transmembrane domain; a 4-1BB, OX-40, or CD28 costimulatory domain; and a CD3ζ primary signaling domain.

In some embodiments, the first and second engineered antigen receptors are a αβ-TCR.

In particular embodiments, the first and second engineered antigen receptors are a DARIC.

In various embodiments, a method of treating a B cell malignancy in a subject is provided comprising: administering to the subject, an effective amount of human CD34⁺ hematopoietic stem and progenitor cells transduced with a lentiviral vector encoding a CAR, wherein the CAR comprises a binding domain that binds one or more target antigens present on a malignant B cell; and administering to the subject, an effective amount of human immune effector cells transduced with the lentiviral vector encoding the CAR; thereby treating the B cell malignancy in the subject.

In particular embodiments, the subject undergoes a myeloablative conditioning regimen, reduced intensity conditioning regimen, or nonmyeloablative conditioning regimen, before administering the CD34⁺ HSPCs and immune effector cells.

In additional embodiments, the human CD34⁺ hematopoietic stem and progenitor cells are allogenic to the subject.

In particular embodiments, the human CD34⁺ hematopoietic stem and progenitor cells are autologous to the subject.

In some embodiments, the human immune effector cells are allogenic to the subject.

In further embodiments, the human immune effector cells are autologous to the subject.

In particular embodiments, the human immune effector cells comprise T cells.

In certain embodiments, the human immune effector cells comprise T cells that express CD3⁺, CD4⁺, CD8⁺, or a combination thereof.

In some embodiments, the human immune effector cells comprise cytotoxic T lymphocytes (CTLs), tumor infiltrating lymphocytes (TILs), and/or helper T cells.

In additional embodiments, the human immune effector cells comprise natural killer (NK) cells or natural killer T (NKT) cells.

In particular embodiments, the B cell malignancy is selected from the group consisting of: leukemias, lymphomas, and myelomas.

In some embodiments, the B cell malignancy is selected from the group consisting of: acute lymphocytic leukemia (ALL), acute myeloid leukemia (AML), myeloblastic, promyelocytic, myelomonocytic, monocytic, erythroleukemia, hairy cell leukemia (HCL), chronic lymphocytic leukemia (CLL), and chronic myeloid leukemia (CML), chronic myelomonocytic leukemia (CMML) and polycythemia vera, Hodgkin lymphoma, nodular lymphocyte-predominant Hodgkin lymphoma, Burkitt lymphoma, small lymphocytic lymphoma (SLL), diffuse large B-cell lymphoma, follicular lymphoma, immunoblastic large cell lymphoma, precursor B-lymphoblastic lymphoma, mantle cell lymphoma, marginal zone lymphoma, mycosis fungoides, anaplastic large cell lymphoma, Sézary syndrome, precursor T-lymphoblastic lymphoma, multiple myeloma, overt multiple myeloma, smoldering multiple myeloma, plasma cell leukemia, non-secretory myeloma, IgD myeloma, osteosclerotic myeloma, solitary plasmacytoma of bone, and extramedullary plasmacytoma.

In further embodiments, the B cell malignancy is multiple myeloma.

In particular embodiments, the one or more target antigens is selected from the group consisting of: BCMA, CD19, CD20, CD22, CD23, CD33, CD37, CD38, CD52, CD79a, CD79b, CD80, and CD123.

In additional embodiments, the one or more target antigens comprises BCMA.

In some embodiments, the one or more target antigens comprises CD19.

In additional embodiments, the one or more target antigens comprises CD20.

In particular embodiments, the one or more target antigens comprises CD22.

In certain embodiments, the one or more target antigens comprises CD23.

In further embodiments, the one or more target antigens comprises CD33.

In particular embodiments, the one or more target antigens comprises CD37.

In additional embodiments, the one or more target antigens comprises CD38.

In some embodiments, the one or more target antigens comprises CD79a.

In certain embodiments, the one or more target antigens comprises CD79b.

In particular embodiments, the one or more target antigens comprises CD123.

In further embodiments, the B cell malignancy expresses a first target antigen and a second target antigen.

In some embodiments, the first and second target antigens are expressed on different malignant B cells.

In particular embodiments, the first and second target antigens are expressed on the same malignant B cells.

In additional embodiments, the CAR comprises: one or more target antigen binding domains; a transmembrane domain; one or more intracellular costimulatory signaling domains; and/or a primary signaling domain.

In some embodiments, the one or more target antigen binding domains is selected from the group consisting of: a Camel Ig, a Llama Ig, an Alpaca Ig, Ig NAR, a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), and a single-domain antibody (sdAb, a camelid VHH, Nanobody).

In particular embodiments, the one or more target antigen binding domains comprises one or more scFvs.

In certain embodiments, the one or more target antigen binding domains comprises one or more VHHs.

In further embodiments, the CAR comprises: an scFv; a CD28 transmembrane domain or a CD8α transmembrane domain; a 4-1BB, OX-40, or CD28 costimulatory domain; and a CD3ζ primary signaling domain.

In some embodiments, the CAR comprises: a VHH; a CD28 transmembrane domain or a CD8α transmembrane domain; a 4-1BB, OX-40, or CD28 costimulatory domain; and a CD3ζ primary signaling domain.

In various embodiments, a method of treating a leukemia, lymphoma, or myeloma in a subject is provided comprising: administering to the subject, an effective amount of autologous human CD34⁺ hematopoietic stem and progenitor cells transduced with a lentiviral vector encoding a CAR, wherein the CAR comprises an scFv or VHH that binds a target antigen; a CD28 transmembrane domain or a CD8α transmembrane domain; a 4-1BB, OX-40, or CD28 costimulatory domain; and a CD3ζ primary signaling domain; and administering to the subject, an effective amount of autologous human immune effector cells transduced with the lentiviral vector encoding the CAR; thereby treating the leukemia, lymphoma, or myeloma in the subject.

In particular embodiments, the subject undergoes a myeloablative conditioning regimen, reduced intensity conditioning regimen, or nonmyeloablative conditioning regimen, before administering the CD34⁺ HSPCs and immune effector cells.

In various embodiments, a method of treating a leukemia, lymphoma, or myeloma in a subject is provided comprising: administering to the subject, an effective amount of allogenic human CD34⁺ hematopoietic stem and progenitor cells transduced with a lentiviral vector encoding a CAR, wherein the CAR comprises an scFv or VHH that binds a target antigen; a CD28 transmembrane domain or a CD8α transmembrane domain; a 4-1BB, OX-40, or CD28 costimulatory domain; and a CD3ζ primary signaling domain; and administering to the subject, an effective amount of allogenic human immune effector cells transduced with the lentiviral vector encoding the CAR; thereby treating the leukemia, lymphoma, or myeloma in the subject.

In particular embodiments, the subject undergoes a myeloablative conditioning regimen, reduced intensity conditioning regimen, or nonmyeloablative conditioning regimen, before administering the CD34⁺ HSPCs and immune effector cells.

In some embodiments, the human immune effector cells comprise T cells.

In particular embodiments, the human immune effector cells comprise T cells that express CD3⁺, CD4⁺, CD8⁺, or a combination thereof.

In additional embodiments, the human immune effector cells comprise cytotoxic T lymphocytes (CTLs), tumor infiltrating lymphocytes (TILs), and/or helper T cells.

In certain embodiments, the human immune effector cells comprise natural killer (NK) cells or natural killer T (NKT) cells.

In particular embodiments, the target antigen is selected from the group consisting of: BCMA, CD19, CD20, CD22, CD23, CD33, CD37, CD38, CD52, CD79a, CD79b, CD80, and CD123.

In further embodiments, the target antigen is BCMA, CD19, CD20, CD22, CD33, CD79a, CD79b, CD80, or CD123.

In various embodiments, a method of preparing a combination adoptive cell therapy product for treating cancer is provided comprising: transducing a population of cells comprising human CD34⁺ hematopoietic stem and progenitor cells with a lentiviral vector encoding a first engineered antigen receptor that binds one or more antigens present on a cancer cell; transducing a population of cells comprising human immune effector cells with a lentiviral vector encoding a second engineered antigen receptor that binds one or more antigens present on a malignant B cell; and formulating the populations of cells for administration to a subject that has, or that has been diagnosed, with a cancer.

In particular embodiments, the subject undergoes a myeloablative conditioning regimen, reduced intensity conditioning regimen, or nonmyeloablative conditioning regimen, before administering the CD34⁺ HSPCs and immune effector cells.

In some embodiments, the human CD34⁺ hematopoietic stem and progenitor cells are allogenic to the subject.

In certain embodiments, the human CD34⁺ hematopoietic stem and progenitor cells are autologous to the subject.

In particular embodiments, the human immune effector cells are allogenic to the subject.

In some embodiments, the human immune effector cells are autologous to the subject.

In additional embodiments, the human immune effector cells comprise T cells, NK cells, or NKT cells.

In particular embodiments, the first and second engineered antigen receptors are selected from the group consisting of: a chimeric antigen receptor (CAR), an αβ T cell receptor (αβ-TCR), a γδ T cell receptor (γδ-TCR), and a dimerizing agent regulated immunoreceptor complex (DARIC).

In further embodiments, the first and second engineered antigen receptors are the same.

In certain embodiments, the first and second engineered antigen receptors are a CAR.

In particular embodiments, the first and second engineered antigen receptors are a αβ-TCR.

In some embodiments, the first and second engineered antigen receptors are a DARIC.

### BRIEF DESCRIPTION OF SEVERAL VIEWS OF THE DRAWINGS

FIG. 1 shows a cartoon of exemplary adoptive cell therapy treatment methods contemplated by the present disclosure.

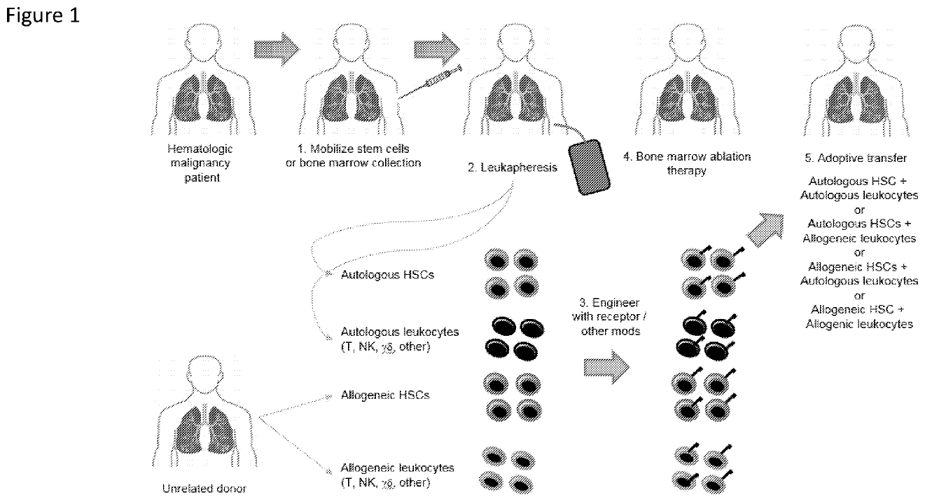

### DETAILED DESCRIPTION

### A. Overview

CAR T cell therapy is increasingly becoming a treatment option for many cancer patients. However, many CAR T cell patients that are initially cleared of disease experience disease recurrence within a year of treatment. Without wishing to be bound to any particular theory, the reasons for disease recurrence include the limited lifespan of CAR T cells in vivo, incomplete tumor clearance, premature CAR T cell clearance, and outgrowth of persistent cancer cells.

The solution to these problems includes adoptive cell therapy that provides both acute and long-term immune effector cell function. Acute effector cell function is provided by administering a subject a population of immune effector cells genetically modified to express an engineered antigen receptor that recognizes a target antigen expressed by a target cell. Long-term effector cell function is provided by administering a subject that has undergone a conditioning regimen (e.g., myeloablative, reduced intensity conditioning, nonmyeloablative conditioning) a population of hematopoietic stem and progenitor cells genetically modified such that the immune effector cell progeny of these cells express an engineered antigen receptor that recognizes a target antigen expressed by a target cell. As the hematopoietic stem cells engraft, they will provide a source of immune effector cells expressing an engineered antigen receptor for the lifetime of the patient. In this way, the methods of adoptive cell therapy contemplated herein solve the problem of disease recurrence caused by incomplete target cell clearance and poor immune effector cell persistence.

In various embodiments, a method of adoptive cell therapy for the treatment of immune system disorder comprises collecting hematopoietic stem and progenitor cells and immune effector cells from a donor, modifying the donor cells so that the donor cells and/or their progeny express an engineered antigen receptor, conditioning a patient, and administering the modified donor cells to the patient.

In preferred embodiments, donor hematopoietic stem and progenitor cells are modified so that the immune effector cell progeny of the cells express a chimeric antigen receptor that binds one or more target antigens expressed by a cancer cell; donor immune effector cells are modified to express a chimeric antigen receptor that binds one or more target antigens expressed by a cancer cell; and the modified donor cells are administered to a conditioned patient. In preferred embodiments, the cancer is a B cell malignancy.

In methods of autologous adoptive cell therapy contemplated herein, the donor and the patient are the same individual, whereas in methods of allogeneic adoptive cell therapy contemplated herein, the donor and the patient are not the same individual. In particular embodiments, the hematopoietic stem and progenitor cells are autologous and the immune effector cells are allogeneic. In particular embodiments, the hematopoietic stem and progenitor cells are allogeneic and the immune effector cells are autologous.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid The Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and CC Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

An “immune disorder” refers to a disease that evokes a response from the immune system. In particular embodiments, the term “immune disorder” refers to a cancer, graft-versus-host disease, an autoimmune disease, or an immunodeficiency. In one embodiment, immune disorders encompass infectious disease.

As used herein, the term “cancer” relates generally to a class of diseases or conditions in which abnormal cells divide without control and can invade nearby tissues.

As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood).

As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor.

As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancers form tumors, but some, e.g., leukemia, do not necessarily form tumors. For those cancers that form tumors, the terms cancer (cell) and tumor (cell) are used interchangeably. The amount of a tumor in an individual is the “tumor burden” which can be measured as the number, volume, or weight of the tumor.

“Graft-versus-host disease” or “GVHD” refers to complications that can occur after cell, tissue, or solid organ transplant. GVHD can occur after a stem cell or bone marrow transplant in which the transplanted donor cells attack the transplant recipient's body. Acute GVHD in humans takes place within about 60 days post-transplantation and results in damage to the skin, liver, and gut by the action of cytolytic lymphocytes. Chronic GVHD occurs later and is a systemic autoimmune disease that affects primarily the skin, resulting in the polyclonal activation of B cells and the hyperproduction of Ig and autoantibodies. Solid-organ transplant graft-versus-host disease (SOT-GVHD) occurs in two forms. The more common type is antibody mediated, wherein antibodies from a donor with blood type O attack a recipient's red blood cells in recipients with blood type A, B, or AB, leading to mild transient, hemolytic anemias. The second form of SOT-GVHD is a cellular type associated with high mortality, wherein donor-derived T cells produce an immunological attack against immunologically disparate host tissue, most often in the skin, liver, gastrointestinal tract, and bone marrow, leading to complications in these organs.

“Graft-versus-leukemia” or “GVL” refer to an immune response to a person's leukemia cells by immune cells present in a donor's transplanted tissue, such as bone marrow or peripheral blood.

An “autoimmune disease” refers to a disease in which the body produces an immunogenic (i.e., immune system) response to some constituent of its own tissue. In other words, the immune system loses its ability to recognize some tissue or system within the body as “self” and targets and attacks it as if it were foreign. Illustrative examples of autoimmune diseases include, but are not limited to: arthritis, inflammatory bowel disease, Hashimoto's thyroiditis, Grave's disease, lupus, multiple sclerosis, rheumatic arthritis, hemolytic anemia, anti-immune thyroiditis, systemic lupus erythematosus, celiac disease, Crohn's disease, colitis, diabetes, scleroderma, psoriasis, and the like.

An “immunodeficiency” means the state of a patient whose immune system has been compromised by disease or by administration of chemicals. This condition makes the system deficient in the number and type of blood cells needed to defend against a foreign substance. Immunodeficiency conditions or diseases are known in the art and include, for example, AIDS (acquired immunodeficiency syndrome), SCID (severe combined immunodeficiency disease), selective IgA deficiency, common variable immunodeficiency, X-linked agammaglobulinemia, chronic granulomatous disease, hyper-IgM syndrome, Wiskott-Aldrich Syndrome (WAS), and diabetes.

An “infectious disease” refers to a disease that can be transmitted from person to person or from organism to organism, and is caused by a microbial or viral agent (e.g., common cold). Infectious diseases are known in the art and include, for example, hepatitis, sexually transmitted diseases (e.g., Chlamydia, gonorrhea), tuberculosis, HIV/AIDS, diphtheria, hepatitis B, hepatitis C, cholera, and influenza.

As used herein, the terms “individual” and “subject” are often used interchangeably and refer to any animal that exhibits a symptom of an immune disorder that can be treated with the methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human subjects, are included. Typical subjects include human patients that have, have been diagnosed with, or are at risk of having an immune disorder.

As used herein, the term “patient” refers to a subject that has been diagnosed with an immune disorder that can be treated with the adoptive cell therapy methods contemplated herein.

As used herein “treatment” or “treating,” includes any beneficial or desirable effect on the symptoms or pathology of a disease or pathological condition, and may include even minimal reductions in one or more measurable markers of the disease or condition being treated, e.g., cancer, GVHD, infectious disease, autoimmune disease, inflammatory disease, and immunodeficiency. Treatment can optionally involve delaying the progression of the disease or condition. “Treatment” does not necessarily indicate complete eradication or cure of the disease or condition, or associated symptoms thereof.

As used herein, “prevent,” and similar words such as “prevention,” “prevented,” “preventing” etc., indicate an approach for preventing, inhibiting, or reducing the likelihood of the occurrence or recurrence of, a disease or condition, e.g., cancer, GVHD, infectious disease, autoimmune disease, inflammatory disease, and immunodeficiency. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As used herein, the phrase “ameliorating at least one symptom of” refers to decreasing one or more symptoms of the disease or condition for which the subject is being treated, e.g., cancer, GVHD, infectious disease, autoimmune disease, inflammatory disease, and immunodeficiency. In particular embodiments, the disease or condition being treated is a cancer, wherein the one or more symptoms ameliorated include, but are not limited to, weakness, fatigue, shortness of breath, easy bruising and bleeding, frequent infections, enlarged lymph nodes, distended or painful abdomen (due to enlarged abdominal organs), bone or joint pain, fractures, unplanned weight loss, poor appetite, night sweats, persistent mild fever, and decreased urination (due to impaired kidney function).

Although full consensus has not been reached within the HCT community, conditioning regimens have been classified as high-dose (myeloablative), reduced-intensity, and nonmyeloablative, following the Reduced-Intensity Conditioning Regimen Workshop, convened by the Center for International Blood and Marrow Transplant Research (CIBMTR) during the Bone Marrow Transplantation Tandem Meeting in 2006.

“Myeloablative” (MA) or “high-dose” regimens generally refers to conditioning that uses alkylating agents and optionally total body irradiation (TBI) that ablates marrow hematopoiesis, does not allow endogenous hematologic recovery, and therefore requires stem cell support. “Reduced Intensity Conditioning” (RIC) generally refers to conditioning that uses alkylating agents and optionally total body irradiation (TBI) and causes a prolonged, but reversible cytopenia and requires stem cell support. RIC regimens differ from myeloablative regimens in that the dose of alkylating agents or TBI is generally reduced by >30%. “Non-MA” (NMA) regimens generally refers to conditioning that causes minimal cytopenia and this type of regimen can be given without stem cell support. The intensity of conditioning regimens can vary substantially, and when selecting the optimal conditioning regimen for any given patient, disease-related factors such as diagnosis and remission status, as well as patient-related factors including age, donor availability, and presence of comorbid conditions, need to be considered. Exemplary conditioning regimens can be found, for example, in Atilla et 42017. Balkan Med J. 34(1): 1-9.

“Autologous,” as used herein, refers to cells from the same subject.

“Allogeneic,” as used herein, refers to cells of the same species that differ genetically to the cell in comparison.

“Syngeneic,” as used herein, refers to cells of a different subject that are genetically identical to the cell in comparison.

“Xenogeneic,” as used herein, refers to cells of a different species to the cell in comparison. In preferred embodiments, the cells are autologous.

### C. Therapeutic Methods

The methods contemplated herein provide improved adoptive cell therapy for use in the prevention, treatment, and amelioration immune disorders, or for preventing, treating, or ameliorating at least one symptom associated with an immune disorder. In particular embodiments, the methods contemplated herein provide improved adoptive cell therapy for use in the prevention, treatment, and amelioration of at least one symptom associated with cancer.

In preferred embodiments, the methods contemplated herein provide improved adoptive cell therapy for use in the treatment of cancer.

The adoptive cell therapy methods contemplated herein provide both acute and long-term immune effector cell function in a subject that has undergone a conditioning regimen. Immune effector cells modified in vitro or ex vivo to express one or more engineered antigen receptors provide acute responses in the subject and durable, long-term responses are provided by the progeny of hematopoietic stem and progenitor cells modified in vitro or ex vivo to express one or more engineered antigen receptors. Without wishing to be bound by any particular theory, it is contemplated that the methods provided herein solve the problem of disease recurrence caused by incomplete disease cell clearance and poor immune effector cell persistence.

In particular embodiments, a subject diagnosed with a cancer is treated using the adoptive cell therapy methods contemplated herein. The adoptive cell therapy methods contemplated in particular embodiments herein are suitable for the treatment of solid and liquid (or hematological) cancers.

In particular embodiments, a subject diagnosed with a solid cancer undergoes a myeloablative conditioning regimen, reduced intensity conditioning regimen, or nonmyeloablative conditioning regimen, and is then treated with the adoptive cell therapies contemplated herein.

Illustrative examples of solid cancers that are suitable for treatment with the adoptive cell therapy methods contemplated in particular embodiments include, but are not limited to, adrenal cancer, adrenocortical carcinoma, anal cancer, appendix cancer, astrocytoma, atypical teratoid/rhabdoid tumor, basal cell carcinoma, bile duct cancer, bladder cancer, bone cancer, brain/CNS cancer, breast cancer, bronchial tumors, cardiac tumors, cervical cancer, cholangiocarcinoma, chondrosarcoma, chordoma, colon cancer, colorectal cancer, craniopharyngioma, ductal carcinoma in situ (DCIS) endometrial cancer, ependymoma, esophageal cancer, esthesioneuroblastoma, Ewing's sarcoma, extracranial germ cell tumor, extragonadal germ cell tumor, eye cancer, fallopian tube cancer, fibrous histiosarcoma, fibrosarcoma, gallbladder cancer, gastric cancer, gastrointestinal carcinoid tumors, gastrointestinal stromal tumor (GIST), germ cell tumors, glioma, glioblastoma, head and neck cancer, hemangioblastoma, hepatocellular cancer, hypopharyngeal cancer, intraocular melanoma, kaposi sarcoma, kidney cancer, laryngeal cancer, leiomyosarcoma, lip cancer, liposarcoma, liver cancer, lung cancer, non-small cell lung cancer, lung carcinoid tumor, malignant mesothelioma, medullary carcinoma, medulloblastoma, menangioma, melanoma, Merkel cell carcinoma, midline tract carcinoma, mouth cancer, myxosarcoma, myelodysplastic syndrome, myeloproliferative neoplasms, nasal cavity and paranasal sinus cancer, nasopharyngeal cancer, neuroblastoma, oligodendroglioma, oral cancer, oral cavity cancer, oropharyngeal cancer, osteosarcoma, ovarian cancer, pancreatic cancer, pancreatic islet cell tumors, papillary carcinoma, paraganglioma, parathyroid cancer, penile cancer, pharyngeal cancer, pheochromocytoma, pinealoma, pituitary tumor, pleuropulmonary blastoma, primary peritoneal cancer, prostate cancer, rectal cancer, retinoblastoma, renal cell carcinoma, renal pelvis and ureter cancer, rhabdomyosarcoma, salivary gland cancer, sebaceous gland carcinoma, skin cancer, soft tissue sarcoma, squamous cell carcinoma, small cell lung cancer, small intestine cancer, stomach cancer, sweat gland carcinoma, synovioma, testicular cancer, throat cancer, thymus cancer, thyroid cancer, urethral cancer, uterine cancer, uterine sarcoma, vaginal cancer, vascular cancer, vulvar cancer, and Wilms Tumor.

In preferred embodiments, the subject is diagnosed with a solid cancer selected from the group consisting of: liver cancer, pancreatic cancer, lung cancer, breast cancer, bladder cancer, brain cancer, bone cancer, thyroid cancer, kidney cancer, and skin cancer.

The adoptive cell therapy methods contemplated in particular embodiments herein are suitable for the treatment of liquid (or hematological) cancers. In particular embodiments, a subject diagnosed with a liquid cancer undergoes a myeloablative conditioning regimen, reduced intensity conditioning regimen, or nonmyeloablative conditioning regimen, and is then treated with the adoptive cell therapies contemplated herein.

Illustrative examples of hematological cancers that are suitable for treatment with the adoptive cell therapy methods contemplated in particular embodiments include, but are not limited to, B cell malignancies such as leukemias, lymphomas, and myelomas. Leukemias, lymphomas, and myelomas suitable for treatment by the adoptive cell therapy methods contemplated in particular embodiments include, but are not limited to, acute lymphocytic leukemia (ALL), acute myeloid leukemia (AML), myeloblastic, promyelocytic, myelomonocytic, monocytic, erythroleukemia, hairy cell leukemia (HCL), chronic lymphocytic leukemia (CLL), and chronic myeloid leukemia (CML), chronic myelomonocytic leukemia (CMML) and polycythemia vera, Hodgkin lymphoma, nodular lymphocyte-predominant Hodgkin lymphoma, Burkitt lymphoma, small lymphocytic lymphoma (SLL), diffuse large B-cell lymphoma, follicular lymphoma, immunoblastic large cell lymphoma, precursor B-lymphoblastic lymphoma, mantle cell lymphoma, marginal zone lymphoma, mycosis fungoides, anaplastic large cell lymphoma, Sézary syndrome, precursor T-lymphoblastic lymphoma, multiple myeloma, overt multiple myeloma, smoldering multiple myeloma, plasma cell leukemia, non-secretory myeloma, IgD myeloma, osteosclerotic myeloma, solitary plasmacytoma of bone, and extramedullary plasmacytoma.

In preferred embodiments, the subject is diagnosed with a B cell malignancy selected from the group consisting of: ALL, AML, CLL, CML, DLBCL, and multiple myeloma.

The adoptive cell therapies contemplated herein comprise hematopoietic stem and progenitor cells (HSPCs) modified so that the immune effector cell progeny of these cells express an engineered antigen receptor. In particular embodiments, HSPCs are modified with vectors encoding engineered antigen receptors selected from the group consisting of: a chimeric antigen receptor (CAR), an αβ T cell receptor (αβ-TCR), a γδ T cell receptor (γδ-TCR), and a dimerizing agent regulated immunoreceptor complex (DARIC). Administration of modified HSPCs to a subject that has undergone a conditioning regimen allows HSPC engraftment and provides a long-term source for immune effector cells to achieve durable, long-lasting responses against an immune disorder, e.g., cancer, in the subject. In preferred embodiments, CD34⁺ hematopoietic stem and progenitor cells are modified to express an engineered antigen receptor by transducing the cells with a vector, e.g., lentiviral vector, comprising a polynucleotide encoding the engineered antigen receptor. The modified CD34⁺ HSPCs are then administered to a subject, e.g., a human, that has undergone conditioning regimen, e.g., a myeloablative conditioning regimen, reduced intensity conditioning regimen, or nonmyeloablative conditioning regimen.

The adoptive cell therapies contemplated herein further comprise immune effector cells modified to express an engineered antigen receptor. In particular embodiments, immune effector cells are modified with vectors encoding engineered antigen receptors selected from the group consisting of: a chimeric antigen receptor (CAR), an αβ T cell receptor (αβ-TCR), a γδ T cell receptor (γδ-TCR), and a dimerizing agent regulated immunoreceptor complex (DARIC). Administration of modified immune effector cells to a subject that has undergone a conditioning regimen provides an immediate source for immune effector cells to achieve robust, short-term responses against an immune disorder, e.g., cancer, in the subject. In preferred embodiments, PBMCs comprising immune effector cells are modified to express an engineered antigen receptor by transducing the cells with a vector, e.g., lentiviral vector, comprising a polynucleotide encoding the engineered antigen receptor. The modified immune effector cells are expanded and then administered to a subject, e.g., a human, that has undergone conditioning regimen, e.g., a myeloablative conditioning regimen, reduced intensity conditioning regimen, or nonmyeloablative conditioning regimen.

In particular embodiments, the adoptive cell therapies contemplated herein comprise hematopoietic stem and progenitor cells (HSPCs) modified so that the immune effector cell progeny of these cells express an engineered antigen receptor and immune effector cells modified to express the same or a different engineered antigen receptor. In particular embodiments, a population of hematopoietic cells comprising HSPCs and a population of PBMCs comprising immune effector cells are modified with vectors encoding one or more engineered antigen receptors selected from the group consisting of: a chimeric antigen receptor (CAR), an αβ T cell receptor (αβ-TCR), a γδ T cell receptor (γδ-TCR), and a dimerizing agent regulated immunoreceptor complex (DARIC). Administration of modified HSPCs and immune effector cells to a subject that has undergone a conditioning regimen provides an immediate source for immune effector cells to achieve robust, short-term responses against an immune disorder, e.g., cancer, in the subject and also allows for HSPC engraftment and provides a long-term source for immune effector cells to achieve durable, long-lasting responses against the immune disorder in the subject. In preferred embodiments, both a population of CD34⁺ hematopoietic stem and progenitor cells and a population of T cells and/or NK cells are modified to express an engineered antigen receptor by transducing the cells with a vector, e.g., lentiviral vector, comprising a polynucleotide encoding one or more engineered antigen receptors. The modified populations of cells are then administered to a subject, e.g., a human, that has undergone conditioning regimen, e.g., a myeloablative conditioning regimen, reduced intensity conditioning regimen, or nonmyeloablative conditioning regimen.

In particular embodiments, the donor HSPCs and donor immune effector cells are autologous to the subject being treated, i.e., they are the patient's own cells. In particular embodiments, the donor HSPCs and donor immune effector cells are allogeneic to the subject being treated, i.e., they are the not the patient's own cells. In particular embodiments, the donor HSPCs are autologous to the subject being treated and the donor immune effector cells are allogeneic to the subject being treated. In particular embodiments, the donor HSPCs are allogeneic to the subject being treated and the donor immune effector cells are autologous to the subject being treated.

In particular embodiments, the adoptive cell therapies contemplated herein are used to treat a subject that has or that has been diagnosed with a B cell malignancy. In particular embodiments, a method of treating a subject with a cancer, e.g., a B cell malignancy, comprises modifying autologous or allogeneic donor hematopoietic stem and progenitor cells (HSPCs) so that immune effector cell progeny of these cells express an engineered antigen receptor and modifying autologous or allogeneic donor immune effector cells to express the same or a different engineered antigen receptor. In particular embodiments, a donor population of hematopoietic cells comprising HSPCs and a donor population of PBMCs comprising immune effector cells are modified with vectors encoding one or more engineered antigen receptors selected from the group consisting of: a chimeric antigen receptor (CAR), an αβ T cell receptor (αβ-TCR), a γδ T cell receptor (γδ-TCR), and a dimerizing agent regulated immunoreceptor complex (DARIC) that bind one or more target antigens. Administration of modified donor HSPCs and immune effector cells to a subject that has undergone a conditioning regimen provides an immediate source for immune effector cells to achieve robust, short-term responses against an immune disorder, e.g., cancer, in the subject and also allows for HSPC engraftment and provides a long-term source for immune effector cells to achieve durable, long-lasting responses against the immune disorder in the subject. In preferred embodiments, both donor populations of CD34⁺ hematopoietic stem and progenitor cells and T cells, natural killer (NK) cells, and/or natural killer T (NKT) cells are modified to express an engineered antigen receptor by transducing the cells with a vector, e.g., lentiviral vector, comprising a polynucleotide encoding one or more engineered antigen receptors. The modified populations of cells are then administered to a subject, e.g., a human, that has undergone conditioning regimen, e.g., a myeloablative conditioning regimen, reduced intensity conditioning regimen, or nonmyeloablative conditioning regimen.

In particular embodiments, a method of treating a subject with a cancer, e.g., a B cell malignancy, comprises modifying autologous or allogeneic donor hematopoietic stem and progenitor cells (HSPCs) and immune effector cells with a vector encoding a chimeric antigen receptor (CAR) that binds one or more target antigens expressed on the cancer cells, and administering the modified donor HSPCs and immune effector cells to a subject that has undergone a myeloablative conditioning regimen, reduced intensity conditioning regimen, or nonmyeloablative conditioning regimen. In preferred embodiments, both donor populations of CD34⁺ hematopoietic stem and progenitor cells and T cells, natural killer (NK) cells, and/or natural killer T (NKT) cells are modified with a vector encoding one or more CARs by transducing the cells with a lentiviral vector comprising a polynucleotide encoding one or more CARs. The modified populations of cells are then administered to a suitably conditioned subject.

In particular embodiments, a method of treating a subject with a cancer, e.g., a B cell malignancy, comprises modifying autologous or allogeneic donor hematopoietic stem and progenitor cells (HSPCs) and immune effector cells with a vector encoding a T cell receptor, an αβ T cell receptor (αβ-TCR) or a γδ T cell receptor (γδ-TCR), that binds one or more target antigens express on the cancer cells, and administering the modified donor HSPCs and immune effector cells to a subject that has undergone a myeloablative conditioning regimen, reduced intensity conditioning regimen, or nonmyeloablative conditioning regimen. In preferred embodiments, both donor populations of CD34⁺ hematopoietic stem and progenitor cells and T cells, natural killer (NK) cells, and/or natural killer T (NKT) cells are modified with a vector encoding one or more αβ-TCRs or γδ-TCRs by transducing the cells with a lentiviral vector comprising a polynucleotide encoding one or more TCRs. The modified populations of cells are then administered to a suitably conditioned subject.

In particular embodiments, a method of treating a subject with a cancer, e.g., a B cell malignancy, comprises modifying autologous or allogeneic donor hematopoietic stem and progenitor cells (HSPCs) and immune effector cells with a vector encoding a dimerizing agent regulated immunoreceptor complex (DARIC) that binds one or more target antigens expressed on the cancer cells, and administering the modified donor HSPCs and immune effector cells to a subject that has undergone a myeloablative conditioning regimen, reduced intensity conditioning regimen, or nonmyeloablative conditioning regimen. In preferred embodiments, both donor populations of CD34⁺ hematopoietic stem and progenitor cells and T cells, natural killer (NK) cells, and/or natural killer T (NKT) cells are modified with a vector encoding one or more DARIC by transducing the cells with a lentiviral vector comprising a polynucleotide encoding one or more DARIC components or DARICs. The modified populations of cells are then administered to a suitably conditioned subject.

The quantity, frequency, and route of administration of the modified HSPCs and immune effector cells will be determined by such factors as the condition of the patient, and the type and severity of the patient's disease, although appropriate dosages may be determined by clinical trials. It is contemplated, that the subject may be administered HSPCs at the same time, before, or after immune effector cells are administered to the subject. It is further contemplated that the HSPCs and immune effector cells may be administered by the same route, preferably, a parental route, more preferably, an intravascular route, and more preferably, intravenously. It is further contemplated that the doses of HSPCs and immune effector cells may be the same or different and depends in part, on the indication, the robustness of cell modification, the conditioning methods, and the health of the subject.

In one embodiment, the amount of HPSCs and/or immune effector cells administered to a subject is at least 0.1×10⁵ cells, at least 0.5×10⁵ cells, at least 1×10⁵ cells, at least 5×10⁵ cells, at least 1×10⁶ cells, at least 0.5×10⁷ cells, at least 1×10⁷ cells, at least 0.5×10⁸ cells, at least 1×10⁸ cells, at least 0.5×10⁹ cells, at least 1×10⁹ cells, at least 2×10⁹ cells, at least 3×10⁹ cells, at least 4×10⁹ cells, at least 5×10⁹ cells, or at least 1×10¹⁰ cells.

In one embodiment, the amount of HPSCs and/or immune effector cells administered to a subject is about 1×10⁷ cells to about 1×10⁹ cells, about 2×10⁷ cells to about 0.9×10⁹ cells, about 3×10⁷ cells to about 0.8×10⁹ cells, about 4×10⁷ cells to about 0.7×10⁹ cells, about 5×10⁷ cells to about 0.6×10⁹ cells, or about 5×10⁷ cells to about 0.5×10⁹ cells.

In one embodiment, the amount of HPSCs and/or immune effector cells administered to a subject is at least 0.1×10⁴ cells/kg of bodyweight, at least 0.5×10⁴ cells/kg of bodyweight, at least 1×10⁴ cells/kg of bodyweight, at least 5×10⁴ cells/kg of bodyweight, at least 1×10⁵ cells/kg of bodyweight, at least 0.5×10⁶ cells/kg of bodyweight, at least 1×10⁶ cells/kg of bodyweight, at least 0.5×10⁷ cells/kg of bodyweight, at least 1×10⁷ cells/kg of bodyweight, at least 0.5×10⁸ cells/kg of bodyweight, at least 1×10⁸ cells/kg of bodyweight, at least 2×10⁸ cells/kg of bodyweight, at least 3×10⁸ cells/kg of bodyweight, at least 4×10⁸ cells/kg of bodyweight, at least 5×10⁸ cells/kg of bodyweight, or at least 1×10⁹ cells/kg of bodyweight.

In one embodiment, the amount of HPSCs and/or immune effector cells administered to a subject is about 1×10⁶ T cells/kg of bodyweight to about 1×10⁸ T cells/kg of bodyweight, about 2×10⁶ T cells/kg of bodyweight to about 0.9×10⁸ T cells/kg of bodyweight, about 3×10⁶ T cells/kg of bodyweight to about 0.8×10⁸ T cells/kg of bodyweight, about 4×10⁶ T cells/kg of bodyweight to about 0.7×10⁸ T cells/kg of bodyweight, about 5×10⁶ T cells/kg of bodyweight to about 0.6×10⁸ T cells/kg of bodyweight, or about 5×10⁶ T cells/kg of bodyweight to about 0.5×10⁸ T cells/kg of bodyweight.

One of ordinary skill in the art would recognize that multiple administrations of the adoptive cell therapies contemplated in particular embodiments may be required to effect the desired result. For example, a population of cells may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

The administration of the compositions contemplated in particular embodiments may be carried out in any convenient manner. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion.

In preferred embodiments, the cell populations contemplated herein are administered to a subject intravenously.

### D. Modified Cells

The adoptive cell therapy methods contemplated in particular embodiments comprise administration of hematopoietic stem and progenitor cells (HSPCs) modified so that the immune effector cell progeny of these cells express an engineered antigen receptor and administration of immune effector cells modified to express an engineered antigen receptor. The HSPCs and immune effector cells may be modified by the same or different methods and with the same or different engineered antigen receptors.

Cells may be non-genetically modified to express one or more engineered antigen receptors contemplated herein, or in particular preferred embodiments, cells may be genetically modified to express one or more engineered antigen receptors contemplated herein. As used herein, the term “genetically engineered” or “genetically modified” refers to the addition of extra genetic material in the form of DNA or RNA into the total genetic material in a cell. The terms, “genetically modified cells,” “modified cells,” and “redirected cells,” are used interchangeably in particular embodiments. An “isolated cell” refers to a cell that has been obtained from an in vivo tissue or organ and is substantially free of extracellular matrix.

In particular embodiments, hematopoietic stem and progenitor cells modified with a vector encoding an engineered antigen receptor are administered to a subject that has an immune disorder, e.g., cancer, to provide a long-term source of immune effector cells; and immune effector cells modified to express the same or a different engineered antigen receptor are administered to the subject to provide a short-term source of immune effector cells.

Hematopoietic stem cells (HSCs) give rise to committed hematopoietic progenitor cells (HPCs) that are capable of generating the entire repertoire of mature blood cells over the lifetime of an organism. The term “hematopoietic stem cell” or “HSC” refers to multipotent stem cells that give rise to the all the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and others known in the art (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827). When transplanted into lethally irradiated animals or humans, hematopoietic stem and progenitor cells (HSPCs) can repopulate the erythroid, neutrophil-macrophage, megakaryocyte and lymphoid hematopoietic cell pool. HSPCs can be autologous/autogeneic (“self”) or non-autologous (“non-self,” e.g., allogeneic, syngeneic or xenogeneic).

Illustrative examples of hematopoietic stem or progenitor cells suitable for use in particular embodiments contemplated herein include hematopoietic cells that are CD34⁺CD38^{Lo}CD90⁺CD45RA⁻, hematopoietic cells that are CD34⁺, CD59⁺, Thy1/CD90⁺, CD38^{Lo/-}, C-kit/CD117⁺, and Lin(−), hematopoietic cells that are CD34⁺, and hematopoietic cells that are CD133⁺. In a particular embodiment, a population of hematopoietic stem and progenitor cells that are genetically modified to express one or more engineered antigen receptors comprises CD133⁺CD90⁺, CD133⁺CD34⁺, or CD133⁺CD90⁺CD34⁺ hematopoietic stem and progenitor cells. In a preferred embodiment, a population of hematopoietic stem and progenitor cells that are genetically modified to express one or more engineered antigen receptors is a CD34⁺ hematopoietic stem and progenitor cell, more preferably a human CD34⁺ hematopoietic stem and progenitor cell.

An “immune effector cell,” is any cell of the immune system that has one or more effector functions (e.g., cytotoxic cell killing activity, secretion of cytokines, induction of ADCC and/or CDC). The illustrative immune effector cells contemplated herein are T lymphocytes, in particular, cytotoxic T cells (CTLs; CD8⁺ T cells), TILs, and helper T cells (HTLs; CD4⁺ T cells). In one embodiment, immune effector cells include natural killer (NK) cells. In one embodiment, immune effector cells include natural killer T (NKT) cells. Immune effector cells can be autologous/autogeneic (“self”) or non-autologous (“non-self,” e.g., allogeneic, syngeneic or xenogeneic).

Illustrative immune effector cells modified to express engineered antigen receptors contemplated herein include T lymphocytes. The terms “T cell” or “T lymphocyte” are art-recognized and are intended to include thymocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), CD4⁺CD8⁺ T cell, CD4⁻CD8⁻ T cell, or any other subset of T cells. Other illustrative populations of T cells suitable for use in particular embodiments include naïve T cells and memory T cells. In preferred embodiments, the T lymphocyte expresses CD62L.

Methods for making the genetically modified cells which express an engineered antigen receptor contemplated herein are provided in particular embodiments. In one embodiment, the method comprises transfecting or transducing a donor HSPC population and a donor immune effector cell population with a vector encoding one or more engineered antigen receptors contemplated herein. In one embodiment, the method comprises selection and/or expansion of the modified cell populations to achieve an effective amount, e.g., a therapeutically effective amount, of cells to be administered to the subject undergoing treatment. In a preferred embodiment, HSPCs are modified and selected for CD34⁺ expression prior to administration to a subject, the modification and selection can be performed in any order; and immune effector cells are modified and expanded in culture prior to administration to the subject.

Cells can be modified in vivo, but in preferred embodiments, cells are modified in vitro or ex vivo.

Adoptive cell therapies contemplated herein comprise cell populations modified to express one or more engineered antigen receptors. In particular embodiments, an engineered antigen receptor is introduced into a cell using a vector that comprises a polynucleotide encoding the engineered antigen receptor. The term “vector” is used herein to refer to a nucleic acid molecule capable of transferring or transporting another nucleic acid molecule. The transferred nucleic acid is generally linked to, e.g., inserted into, the vector nucleic acid molecule. A vector may include sequences that direct autonomous replication in a cell or may include sequences sufficient to allow integration into host cell DNA.

In particular embodiments, the vector is a non-viral vector, including, but not limited to plasmids (e.g., DNA plasmids or RNA plasmids), transposons, cosmids, and bacterial artificial chromosomes.

Illustrative methods of non-viral delivery of polynucleotides contemplated in particular embodiments include, but are not limited to: electroporation, sonoporation, lipofection, microinjection, biolistics, virosomes, liposomes, immunoliposomes, nanoparticles, polycation or lipid:nucleic acid conjugates, naked DNA, artificial virions, DEAE-dextran-mediated transfer, gene gun, and heat-shock.

In preferred embodiments, viral vectors are used to modify HSPCs and immune effector cells to express one or more engineered antigen receptors contemplated herein.

Illustrative examples of viral vector systems suitable for introducing a polynucleotide encoding an engineered antigen receptor into an HSPC or immune effector cell include, but are not limited to adeno-associated virus (AAV), retrovirus, herpes simplex virus, adenovirus, vaccinia virus vectors for gene transfer.

In various embodiments, HSPCs and immune effector cells are modified by transducing the cell with a recombinant adeno-associated virus (rAAV), comprising one or more polynucleotides encoding the one or more engineered antigen receptors.

AAV is a small (˜26 nm) replication-defective, primarily episomal, non-enveloped virus. AAV can infect both dividing and non-dividing cells and may incorporate its genome into that of the host cell. Recombinant AAV (rAAV) are typically composed of, at a minimum, a transgene and its regulatory sequences, and 5′ and 3′ AAV inverted terminal repeats (ITRs). The ITR sequences are about 145 bp in length. In particular embodiments, the rAAV comprises ITRs and capsid sequences isolated from AAV1, AAV2, AAV3, AAV4, AAV5, AAV6, AAV7, AAV8, AAV9, or AAV10.

In some embodiments, a chimeric rAAV is used and the ITR sequences are isolated from one AAV serotype and the capsid sequences are isolated from a different AAV serotype. For example, a rAAV with ITR sequences derived from AAV2 and capsid sequences derived from AAV6 is referred to as AAV2/AAV6. In particular embodiments, the rAAV vector may comprise ITRs from AAV2, and capsid proteins from any one of AAV1, AAV2, AAV3, AAV4, AAV5, AAV6, AAV7, AAV8, AAV9, or AAV10. In a preferred embodiment, the rAAV comprises ITR sequences derived from AAV2 and capsid sequences derived from AAV6. In a preferred embodiment, the rAAV comprises ITR sequences derived from AAV2 and capsid sequences derived from AAV2.

Construction of rAAV vectors, production, and purification thereof have been disclosed, e.g., in U.S. Pat. Nos. 9,169,494; 9,169,492; 9,012,224; 8,889,641; 8,809,058; and 8,784,799, each of which is incorporated by reference herein, in its entirety.

In various embodiments, HSPCs and immune effector cells are modified by transducing the cell with a retrovirus, e.g., lentivirus, comprising one or more polynucleotides encoding the one or more engineered antigen receptors.

As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

In various embodiments, a lentiviral vector contemplated herein comprises one or more LTRs, and one or more, or all, of the following accessory elements: a cPPT/FLAP, a Psi (T) packaging signal, an export element, a promoter active in immune effector cells operably linked to a multivalent CAR, poly (A) sequences, and may optionally comprise a WPRE or HPRE, an insulator element, a selectable marker, and a cell suicide gene, as discussed elsewhere herein.

In particular embodiments, lentiviral vectors contemplated herein may be integrative or non-integrating or integration defective lentiviruses. As used herein, the term “integration defective lentivirus” or “IDLV” refers to a lentivirus having an integrase that lacks the capacity to integrate the viral genome into the genome of the host cells. Integration-incompetent viral vectors have been described in patent application WO 2006/010834, which is herein incorporated by reference in its entirety.

Illustrative mutations in the HIV-1 pol gene suitable to reduce integrase activity include, but are not limited to: H12N, H12C, H16C, H16V, S81 R, D41A, K42A, H51A, Q53C, D55V, D64E, D64V, E69A, K71A, E85A, E87A, D116N, D1161, D116A, N120G, N1201, N120E, E152G, E152A, D35E, K156E, K156A, E157A, K159E, K159A, K160A, R166A, D167A, E170A, H171A, K173A, K186Q, K186T, K188T, E198A, R199c, R199T, R199A, D202A, K211A, Q214L, Q216L, Q221 L, W235F, W235E, K236S, K236A, K246A, G247W, D253A, R262A, R263A and K264H.

In one embodiment, the HIV-1 integrase deficient pol gene comprises a D64V, D116I, D116A, E152G, or E152A mutation; D64V, D116I, and E152G mutations; or D64V, D116A, and E152A mutations.

In one embodiment, the HIV-1 integrase deficient pol gene comprises a D64V mutation.

The term “long terminal repeat (LTR)” refers to domains of base pairs located at the ends of retroviral DNAs which, in their natural sequence context, are direct repeats and contain U3, R and U5 regions.

As used herein, the term “FLAP element” or “cPPT/FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173. In another embodiment, a lentiviral vector contains a FLAP element with one or more mutations in the cPPT and/or CTS elements. In yet another embodiment, a lentiviral vector comprises either a cPPT or CTS element. In yet another embodiment, a lentiviral vector does not comprise a cPPT or CTS element.

As used herein, the term “packaging signal” or “packaging sequence” refers to psi [Ψ] sequences located within the retroviral genome which are required for insertion of the viral RNA into the viral capsid or particle, see e.g., Clever et al., 1995. J. of Virology, Vol. 69, No. 4; pp. 2101-2109.

The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE).

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766).

Lentiviral vectors preferably contain several safety enhancements as a result of modifying the LTRs. “Self-inactivating” (SIN) vectors refers to replication-defective vectors, e.g., in which the right (3′) LTR enhancer-promoter region, known as the U3 region, has been modified (e.g., by deletion or substitution) to prevent viral transcription beyond the first round of viral replication. An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters.

The terms “pseudotype” or “pseudotyping” as used herein, refer to a virus whose viral envelope proteins have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4⁺ presenting cells.

In certain embodiments, lentiviral vectors are produced according to known methods. See e.g., Kutner et al., BMC Biotechnol. 2009; 9:10. doi: 10.1186/1472-6750-9-10; Kutner et al. Nat. Protoc. 2009; 4(4):495-505. doi: 10.1038/nprot.2009.22.

According to certain specific embodiments contemplated herein, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid contemplated herein.

In various embodiments, HSPCs and immune effector cells are modified by transducing the cell with an adenovirus comprising one or more polynucleotides encoding the one or more engineered antigen receptors.

Adenoviral based vectors are capable of very high transduction efficiency in many cell types and do not require cell division. With such vectors, high titer and high levels of expression have been obtained. This vector can be produced in large quantities in a relatively simple system. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Generation and propagation of the current adenovirus vectors, which are replication deficient, may utilize a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991). Adenovirus vectors have been used in eukaryotic gene expression (Levrero et al., 1991; Gomez-Foix et al., 1992) and vaccine development (Grunhaus & Horwitz, 1992; Graham & Prevec, 1992). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993). An example of the use of an Ad vector in a clinical trial involved polynucleotide therapy for antitumor immunization with intramuscular injection (Sterman et al., Hum. Gene Ther. 7:1083-9 (1998)).

In various embodiments, HSPCs and immune effector cells are modified by transducing the cell with a herpes simplex virus, e.g., HSV-1, HSV-2, comprising one or more polynucleotides encoding the one or more engineered antigen receptors.

The mature HSV virion consists of an enveloped icosahedral capsid with a viral genome consisting of a linear double-stranded DNA molecule that is 152 kb. In one embodiment, the HSV based viral vector is deficient in one or more essential or non-essential HSV genes. In one embodiment, the HSV based viral vector is replication deficient. Most replication deficient HSV vectors contain a deletion to remove one or more intermediate-early, early, or late HSV genes to prevent replication. For example, the HSV vector may be deficient in an immediate early gene selected from the group consisting of: ICP4, ICP22, ICP27, ICP47, and a combination thereof. Advantages of the HSV vector are its ability to enter a latent stage that can result in long-term DNA expression and its large viral DNA genome that can accommodate exogenous DNA inserts of up to 25 kb. HSV-based vectors are described in, for example, U.S. Pat. Nos. 5,837,532, 5,846,782, and 5,804,413, and International Patent Applications WO 91/02788, WO 96/04394, WO 98/15637, and WO 99/06583, each of which are incorporated by reference herein in its entirety.

### E. Engineered Antigen Receptors

Hematopoietic stem and progenitor cells and immune effectors cells used in the adoptive cell therapies contemplated herein are modified with a vector encoding an engineered antigen receptor that recognizes or binds a target antigen that is expressed on a target cell. In particular embodiments, the engineered antigen receptor is an engineered αβ T cell receptor (αβTCR), an engineered γδ TCR, a chimeric antigen receptor (CAR), or a dimerizing agent regulated immunoreceptor complex (DARIC) or components thereof.

In particular embodiments, an engineered antigen receptor is designed to bind one or more target antigens selected from the group consisting of: tumor associated antigens (TAA), tumor specific antigens (TSA), NKG2D ligands, γδ T cell receptor (TCR) ligands, and αβ TCR ligands.

In particular embodiments, an engineered antigen receptor is designed to bind one or more target antigens selected from the group consisting of: alpha folate receptor (FRα), αvβ6 integrin, B cell maturation antigen (BCMA), B7-H3 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IL-10Rα, IL-13Rα2, Kappa, cancer/testis antigen 2 (LAGE-1A), Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), trophoblast glycoprotein (TPBG), UL16-binding protein (ULBP) 1, ULBP2, ULBP3, ULBP4, ULBP5, ULBP6, vascular endothelial growth factor receptor 2 (VEGFR2), and Wilms tumor 1 (WT-1).

In particular embodiments, an engineered antigen receptor is designed to bind one or more target antigens expressed on a B cell malignancy, the one or more target antigens selected from the group consisting of: BCMA, CD19, CD20, CD22, CD23, CD33, CD37, CD38, CD52, CD79a, CD79b, CD80, C123, and HLA-DR.

1. Engineered TCRs

Naturally occurring T cell receptors comprise two subunits, an alpha chain and a beta chain subunit (αβTCR), or a gamma chain and a delta chain subunit (γδTCR), each of which is a unique protein produced by a recombination event in each T cell's genome. Libraries of TCRs may be screened for their selectivity to particular target antigens. In this manner, natural TCRs, which have a high-avidity and reactivity toward target antigens may be selected, cloned, and subsequently introduced into a population of HSPCs and immune effector cells used for adoptive cell therapy. In one embodiment, the TCR is an αβTCR. In one embodiment, the TCR is a γδTCR.

The nucleic acids encoding engineered TCRs are preferably isolated from their natural context in a (naturally-occurring) chromosome of a T cell, and can be incorporated into suitable vectors as described elsewhere herein. Both the nucleic acids and the vectors comprising them can be transferred into a cell. The progeny of modified HSPCs and immune effector cells are then able to express one or more chains of a TCR encoded by the transduced nucleic acid or nucleic acids. In preferred embodiments, the engineered TCR is an exogenous TCR because it is introduced into cells that do not normally express the particular TCR. The essential aspect of the engineered TCRs is that it has high avidity for a tumor antigen presented by a major histocompatibility complex (MHC) or similar immunological component. In contrast to engineered TCRs, CARs are engineered to bind target antigens in an MHC independent manner.

The TCR can be expressed with additional polypeptides attached to the amino-terminal or carboxyl-terminal portion of the alpha chain or beta chain of a TCR, or of the gamma chain or delta chain of a TCR so long as the attached additional polypeptide does not interfere with the ability of the alpha chain or beta chain to form a functional T cell receptor and the MHC dependent antigen recognition.

Target antigens that are recognized by the engineered TCRs contemplated in particular embodiments include, but are not limited to cancer antigens, including antigens on both hematological cancers and solid tumors. Illustrative target antigens that can be targeted by TCRs contemplated herein include, but are not limited to FRα, αᵥβ₆ integrin, BCMA, CD276, B7-H6, CAIX, CD16, CD19, CD20, CD22, CD30, CD33, CD37, CD38, CD44, CD44v6, CD44v7/8, CD70, CD79a, CD79b, CD123, CD133, CD138, CD171, CEA, CLL-1, CS-1, CSPG4, CTAGE1, EGFR, EGFRvIII, EGP2, EGP40, EPCAM, EPHA2, FAP, FCRL5, AchR, GD2, GD3, GPC3, HER2, IL-11Rα, IL-13Rα2, LAGE-1A, Lambda, LeY, L1-CAM, MAGE-A1, MAGE-A3, MAGE-A4, MAGE-A6, MAGEA10, MelanA or MART1, MSLN, MUC1, MUC16, MICA, MICB, NCAM, NY-ESO-1, PLAC1, PRAME, PSCA, PSMA, ROR1, SSX2, Survivin, TAG72, TEM1/CD248, TEM7R, TPBG, ULBP1, ULBP2, ULBP3, ULBP4, ULBP5, ULBP6, VEGFR2, and WT-1.

2. Chimeric Antigen Receptors (CARs)

In various embodiments, HSPCs and immune effector cells are modified with a vector encoding one or more chimeric antigen receptors (CARs) that redirect cytotoxicity toward a target antigen that is expressed on a target cell. CARs are molecules that combine antibody-based specificity for a target antigen with a T cell receptor-activating intracellular domain to generate a chimeric protein that exhibits a specific anti-tumor cellular immune activity. As used herein, the term, “chimeric,” describes being composed of parts of different proteins or DNAs from different origins. In one embodiment, cells are engineered by introducing a polynucleotide or vector encoding a CAR.

In various embodiments, a CAR comprises an extracellular antigen binding domain that binds to a specific target antigen, a transmembrane domain and one or more intracellular signaling domains. The main characteristic of CARs is their ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific coreceptors.

In particular embodiments, CARs comprise an extracellular antigen binding domain that specifically binds to a target polypeptide. An antigen binding domain includes any naturally occurring, synthetic, semi-synthetic, or recombinantly produced binding partner for a biological molecule of interest.

In particular embodiments, the extracellular binding domain comprises an antibody or antigen binding fragment thereof.

In particular embodiments, the extracellular binding domain comprises an antibody or antigen binding fragment thereof is selected from the group consisting of: a Camel Ig, a Llama Ig, an Alpaca Ig, Ig NAR, a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), and a single-domain antibody (sdAb, a camelid VHH, Nanobody).

In one preferred embodiment, the binding domain is an scFv.

In another preferred embodiment, the binding domain is a camelid antibody, e.g., VHH.

In particular embodiments, the CAR comprises an extracellular domain that binds an antigen selected from the group consisting of: FRα, aᵥβ₆ integrin, BCMA, CD276, B7-H6, CAIX, CD16, CD19, CD20, CD22, CD30, CD33, CD37, CD38, CD44, CD44v6, CD44v7/8, CD70, CD79a, CD79b, CD123, CD133, CD138, CD171, CEA, CLL-1, CS-1, CSPG4, CTAGE1, EGFR, EGFRvIII, EGP2, EGP40, EPCAM, EPHA2, FAP, FCRL5, AchR, GD2, GD3, GPC3, HER2, IL-11Rα, IL-13Rα2, LAGE-1A, Lambda, LeY, L1-CAM, MAGE-A1, MAGE-A3, MAGE-A4, MAGE-A6, MAGEA10, MelanA or MART1, MSLN, MUC1, MUC16, MICA, MICB, NCAM, NY-ESO-1, PLAC1, PRAME, PSCA, PSMA, ROR1, SSX2, Survivin, TAG72, TEM1/CD248, TEM7R, TPBG, ULBP1, ULBP2, ULBP3, ULBP4, ULBP5, ULBP6, VEGFR2, and WT-1.

In particular embodiments, a CAR comprises a spacer domain. In one embodiment, the spacer domain comprises the CH2 and CH3 of IgG1, IgG4, or IgD.

In particular embodiments, a CAR comprises a hinge region. Illustrative hinge domains suitable for use in the CARs described herein include the hinge region derived from the extracellular regions of type 1 membrane proteins such as CD8α, and CD4, which may be wild-type hinge regions from these molecules or may be altered. In another embodiment, the hinge domain comprises a CD8α hinge region.

The transmembrane (TM) domain of the CAR fuses the extracellular binding portion and intracellular signaling domain and anchors the CAR to the plasma membrane of the immune effector cell. The TM domain may be derived either from a natural, synthetic, semi-synthetic, or recombinant source.

Illustrative TM domains may be derived from (i.e., comprise) at least the transmembrane region(s) of the alpha, beta, gamma, or delta chain of the T-cell receptor, CD3δ, CD3ε, CD3γ, CD3ζ, CD4, CD5, CD8α, CD9, CD 16, CD22, CD27, CD28, CD33, CD37, CD45, CD64, CD80, CD86, CD 134, CD137, CD152, CD154, AMN, and PD-1.

In one embodiment, a CAR comprises a TM domain derived from CD8α. In another embodiment, a CAR contemplated herein comprises a TM domain derived from CD8α and a short oligo- or polypeptide linker, preferably between 1, 2, 3, 4, 5, 6, 7, 8, 9, or 10 amino acids in length that links the TM domain and the intracellular signaling domain of the CAR. A glycine-serine linker provides a particularly suitable linker.

In preferred embodiments, a CAR comprises an intracellular signaling domain that comprises one or more “co-stimulatory signaling domains” and a “primary signaling domain.”

Primary signaling domains that act in a stimulatory manner may contain signaling motifs which are known as immunoreceptor tyrosine-based activation motifs or ITAMs.

Illustrative examples of ITAM containing primary signaling domains suitable for use in CARs contemplated in particular embodiments include those derived from FcRγ, FcRβ, CD3γ, CD3δ, CD3ε, CD3ζ, CD22, CD79a, CD79b, and CD66d. In particular preferred embodiments, a CAR comprises a CD3ζ primary signaling domain and one or more co-stimulatory signaling domains. The intracellular primary signaling and co-stimulatory signaling domains may be linked in any order in tandem to the carboxyl terminus of the transmembrane domain.

In particular embodiments, a CAR comprises one or more co-stimulatory signaling domains to enhance the efficacy and expansion of T cells expressing CAR receptors.

Illustrative examples of such co-stimulatory molecules suitable for use in CARs contemplated in particular embodiments include TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70. In one embodiment, a CAR comprises one or more co-stimulatory signaling domains selected from the group consisting of CD28, CD137, and CD134, and a CD3ζ primary signaling domain.

In various embodiments, the CAR comprises: an extracellular domain that binds an antigen selected from the group consisting of: BCMA, CD19, CD20, CD22, CD33, CD79a, CD79b, or CD123; a transmembrane domain isolated from a polypeptide selected from the group consisting of: CD4, CD8α, CD154, and PD-1; one or more intracellular co-stimulatory signaling domains isolated from a polypeptide selected from the group consisting of: CD28, CD134, and CD137; and a signaling domain isolated from a polypeptide selected from the group consisting of: FcRγ, FcRβ, CD3γ, CD3δ, CD3ε, CD3ζ, CD22, CD79a, CD79b, and CD66d.

3. DARIC Receptors

In particular embodiments, immune effector cells are modified with a vector encoding one or more DARIC receptor components that recognize or bind a target antigen that is expressed on a target cell. As used herein, the term “DARIC receptor” refers to one or more non-naturally occurring polypeptides that transduces an immunostimulatory signal in an immune effector cell upon exposure to a multimerizing agent or bridging factor, e.g., stimulating immune effector cell activity and function, increasing production and/or secretion of proinflammatory cytokines. In preferred embodiments, the DARIC receptor is a multi-chain receptor comprising a DARIC signaling component and a DARIC binding component.

A “DARIC signaling component” or “DARIC signaling polypeptide” refers to a polypeptide comprising one or more multimerization domains, a transmembrane domain, and an intracellular signaling domain. In particular embodiments, the DARIC signaling component comprises a multimerization domain, a transmembrane domain, a co-stimulatory domain and/or a primary signaling domain.

Illustrative examples of multimerization domains suitable for use in particular DARIC signaling components contemplated herein include, but are not limited to, an FK506 binding protein (FKBP) polypeptide or variants thereof, or an FKBP-rapamycin binding (FRB) polypeptide or variants thereof. In particular preferred embodiments, a DARIC signaling component comprises an FRB polypeptide comprising a T2098L mutation, or variant thereof. In certain preferred embodiments, a DARIC signaling component comprises an FKBP12 polypeptide or variant thereof.

Illustrative examples of transmembrane domains suitable for use in particular DARIC signaling components contemplated herein include, but are not limited to, the transmembrane region(s) of the alpha, beta, gamma, or delta chain of a T-cell receptor, CD3ε, CD3ζ, CD4, CD5, CD8α, CD9, CD 16, CD22, CD27, CD28, CD33, CD37, CD45, CD64, CD71, CD80, CD86, CD 134, CD137, CD152, CD 154, AMN, and PD1. In particular preferred embodiments, a DARIC signaling component comprises a CD8α transmembrane domain. In certain preferred embodiments, an DARIC signaling component comprises a CD4 transmembrane domain.

In various preferred embodiments, a short oligo- or poly-peptide linker, preferably between 1, 2, 3, 4, 5, 6, 7, 8, 9, or 10 amino acids in length links the transmembrane domain and the intracellular signaling domain. A glycine-serine based linker provides a particularly suitable linker.

DARIC signaling components contemplated herein comprise one or more intracellular signaling domains. In one embodiment, a DARIC signaling component comprises one or more co-stimulatory signaling domains and/or a primary signaling domain. In one embodiment, the intracellular signaling domain comprises an immunoreceptor tyrosine activation motif (ITAM).

Illustrative examples of ITAM containing primary signaling domains that are suitable for use in particular DARIC signaling components contemplated herein include, but are not limited to those derived from FcRγ, FcRβ, CD3γ, CD3δ, CD3ε, CD3ζ, CD22, CD79a, CD79b, and CD66d. In particular preferred embodiments, an NKG2D DARIC signaling component comprises a CD3ζ primary signaling domain and one or more co-stimulatory signaling domains. The primary signaling and co-stimulatory signaling domains may be linked in any order in tandem to the carboxyl terminus of the transmembrane domain.

Illustrative examples of such co-stimulatory molecules suitable for use in particular DARIC signaling components contemplated herein include, but are not limited to, TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70. In particular embodiments, a DARIC signaling component comprises one or more co-stimulatory signaling domains selected from the group consisting of CD28, CD137, and CD134. In particular embodiments, a DARIC signaling component comprises one or more co-stimulatory signaling domains selected from the group consisting of CD28, CD137, and CD134, and a CD3ζ primary signaling domain. In particular preferred embodiments, a DARIC signaling component comprises a CD137 co-stimulatory domain and a CD3ζ primary signaling domain.

In certain preferred embodiments, a DARIC signaling component comprises an FRB T2098L multimerization domain, a CD8α transmembrane domain, a CD137 co-stimulatory domain and a CD3ζ primary signaling domain.

A “DARIC binding component” or “DARIC binding polypeptide” refers to a polypeptide comprising an extracellular antigen binding domain, one or more multimerization domains, a transmembrane domain, and an intracellular signaling domain.

In particular embodiments, the extracellular binding domain of a DARIC binding component is an antibody or antigen binding fragment thereof including, but not limited to a Camel Ig, Ig NAR, Fab fragments, Fab′ fragments, F(ab)′2 fragments, F(ab)′3 fragments, Fv, single chain variable fragments (“scFv”), bis-scFv, (scFv)₂, minibodies, diabodies, triabodies, tetrabodies, disulfide stabilized Fv proteins (“dsFv”), or a single-domain antibody (sdAb, Nanobody.

In particular embodiments, the DARIC binding component comprises an extracellular domain that binds an antigen selected from the group consisting of: FRα, αᵥβ₆ integrin, BCMA, CD276, B7-H6, CAIX, CD16, CD19, CD20, CD22, CD30, CD33, CD37, CD38, CD44, CD44v6, CD44v7/8, CD70, CD79a, CD79b, CD123, CD133, CD138, CD171, CEA, CLL-1, CS-1, CSPG4, CTAGE1, EGFR, EGFRvII, EGP2, EGP40, EPCAM, EPHA2, FAP, FCRL5, AchR, GD2, GD3, GPC3, HER2, IL-11Rα, IL-13Rα2, LAGE-1A, Lambda, LeY, L1-CAM, MAGE-A1, MAGE-A3, MAGE-A4, MAGE-A6, MAGEA10, MelanA or MART1, MSLN, MUC1, MUC16, MICA, MICB, NCAM, NY-ESO-1, PLAC1, PRAME, PSCA, PSMA, ROR1, SSX2, Survivin, TAG72, TEM1/CD248, TEM7R, TPBG, ULBP1, ULBP2, ULBP3, ULBP4, ULBP5, ULBP6, VEGFR2, and WT-1.

Illustrative examples of multimerization domains suitable for use in particular DARIC binding components contemplated herein include, but are not limited to, an FK506 binding protein (FKBP) polypeptide or variants thereof, or an FKBP-rapamycin binding (FRB) polypeptide or variants thereof. In particular preferred embodiments, a DARIC signaling component comprises an FKBP12 polypeptide or variant thereof. In certain preferred embodiments, a DARIC signaling component comprises an FRB polypeptide comprising a T2098L mutation, or variant thereof.

Illustrative examples of transmembrane domains suitable for use in particular DARIC binding components contemplated herein include, but are not limited to, the transmembrane region(s) of the alpha, beta, gamma, or delta chain of a T-cell receptor, CD3ε, CD3ζ, CD4, CD5, CD8α, CD9, CD 16, CD22, CD27, CD28, CD33, CD37, CD45, CD64, CD71, CD80, CD86, CD 134, CD137, CD152, CD 154, AMN, and PD1. In particular preferred embodiments, a DARIC binding component comprises a CD4 transmembrane domain. In certain preferred embodiments, a DARIC binding component comprises a CD8α transmembrane domain.

In various preferred embodiments, a short oligo- or poly-peptide linker, preferably between 1, 2, 3, 4, 5, 6, 7, 8, 9, or 10 amino acids in length links the transmembrane domain and the intracellular signaling domain. A glycine-serine based linker provides a particularly suitable linker.

In particular embodiments, the DARIC binding components contemplated herein comprise a signal peptide, e.g., secretion signal peptide, and do not comprise a transmembrane domain. Illustrative examples of signal peptides suitable for use in particular DARIC binding components include, but are not limited to an IgG1 heavy chain signal polypeptide, an Igκ light chain signal polypeptide, a CD8α signal polypeptide, or a human GM-CSF receptor alpha signal polypeptide. In various preferred embodiments, a DARIC binding component comprises a CD8α signal polypeptide.

In particular preferred embodiments, a DARIC binding component comprises an scFv or single domain antibody that binds a target antigen, an FKBP12 multimerization domain, and a CD4 transmembrane domain.

Bridging factors contemplated herein mediate or promote the association of DARIC signaling components with DARIC binding components through the component multimerization domains. A bridging factor associates with and is disposed between the multimerization domains to promote association of a DARIC signaling component and a DARIC binding component. In the presence of a bridging factor, the binding component and the signaling component associate and initiate immune effector cell activity against a target cell when the DARIC binding polypeptide is bound to a target antigen on the target cell. In the absence of a bridging factor, the DARIC binding component does not associate with the DARIC signaling component.

In particular embodiments, a DARIC signaling component and a DARIC binding component comprise one or more FRB and/or FKBP multimerization domains or variants thereof. In certain embodiments, a DARIC signaling component comprises an FRB multimerization domain or variant thereof and a DARIC binding component comprises an FKBP multimerization domain or variant thereof. In particular preferred embodiments, a DARIC signaling component comprises an FRB T2098L multimerization domain or variant thereof and a DARIC binding component comprises an FKBP12 or FKBP12 F36V multimerization domain or variant thereof.

Illustrative examples of bridging factors suitable for use in particular embodiments contemplated herein include, but are not limited to, AP1903, AP20187, AP21967 (also known as C-16-(S)-7-methylindolerapamycin), everolimus, novolimus, pimecrolimus, ridaforolimus, tacrolimus, temsirolimus, umirolimus, and zotarolimus. In particular preferred embodiments, the bridging factor is AP21967. In certain preferred embodiments, the bridging factor is sirolimus (rapamycin).

### F. Compositions and Formulations

The compositions contemplated herein may comprise one or more engineered antigen receptors, polynucleotides, vectors comprising same, genetically modified immune effector cells, etc. Compositions include, but are not limited to pharmaceutical compositions. A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In particular embodiments, compositions comprise an amount of modified HSPCS or immune effector cells contemplated herein.

As used herein, the term “amount” refers to “an amount effective” or “an effective amount” of a therapeutic cell, to achieve a beneficial or desired prophylactic or therapeutic result, including clinical results.

A “prophylactically effective amount” refers to an amount of a therapeutic cell effective to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a therapeutic cell may vary according to factors such as the disease state, age, sex, and weight of the individual, and the ability of the composition to elicit a desired response in the individual. A therapeutically effective amount is also one in which any toxic or detrimental effects of a therapeutic cell are outweighed by the therapeutically beneficial effects. The term “therapeutically effective amount” includes an amount that is effective to “treat” a subject (e.g., a patient).

Generally, compositions comprising the cells contemplated herein may be utilized in the treatment and prevention of diseases that arise in individuals who are immunocompromised. In particular, compositions contemplated herein are used in the treatment of cancer.

In particular embodiments, pharmaceutical compositions comprise an amount of genetically modified cells, in combination with one or more pharmaceutically or physiologically acceptable carriers, diluents or excipients.

Pharmaceutical compositions comprising a cell population, such as HSPCs or immune effector cells, may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In particular embodiments, compositions are preferably formulated for nasal, oral, enteral, or parenteral administration, e.g., intravascular (intravenous or intraarterial), intraperitoneal or intramuscular administration.

The liquid pharmaceutical compositions, whether they be solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In one embodiment, the cell compositions contemplated herein are formulated in a pharmaceutically acceptable cell culture medium. Such compositions are suitable for administration to human subjects. In particular embodiments, the pharmaceutically acceptable cell culture medium is a serum free medium.

Serum-free medium has several advantages over serum containing medium, including a simplified and better defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein.

Illustrative examples of serum-free media used in particular compositions includes but is not limited to QBSF-60 (Quality Biological, Inc.), StemPro-34 (Life Technologies), and X-VIVO 10.

In one preferred embodiment, compositions comprising HSPCs and immune effector cells contemplated herein are formulated in a solution comprising PlasmaLyte A.

In another preferred embodiment, compositions comprising HSPCs and immune effector cells contemplated herein are formulated in a solution comprising a cryopreservation medium. For example, cryopreservation media with cryopreservation agents may be used to maintain a high cell viability outcome post-thaw. Illustrative examples of cryopreservation media used in particular compositions includes, but is not limited to, CryoStor CS10, CryoStor CS5, and CryoStor CS2.

In a more preferred embodiment, compositions comprising HSPCs and immune effector cells contemplated herein are formulated in a solution comprising 50:50 PlasmaLyte A to CryoStor CS10.

In a particular embodiment, compositions comprise an effective amount of HSPCs and immune effector cells, alone or in combination with one or more therapeutic agents. Thus, the HSPCs and immune effector cell compositions may be administered alone or in combination with other known cancer treatments, such as radiation therapy, chemotherapy, transplantation, immunotherapy, hormone therapy, photodynamic therapy, etc.

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Adoptive Cell Therapy

### Introduction

Recent studies have shown that ˜40% of pediatric ALL patients who are treated with anti-CD19 chimeric antigen receptor (CAR) T cells will experience disease relapse (Maude, N Engl J Med, 2018). Although there have been several mechanisms proposed for the relapse including lymphoblast downregulation of CD19 and robust immunosuppression by the bone marrow microenvironment, inadequate persistence of anti-CD19 CAR T cells has also been implicated. In patients who experience a relapse following anti-CD19 CAR therapy, repeat treatment with a second CAR is beneficial (Fry, Nature Med, 2018).

One method to provide patients with a continuous supply of fresh CAR T cells is to transduce hematopoietic stem cells with a lentiviral vector (LVV) encoding the CAR. Administering the patient transduced HSCs will result in long-term CAR expression in both the lymphoid and myeloid progeny. Lymphoid progenitor cells harboring the CAR will circulate to the thymus and differentiate into mature CAR T cells, thus providing the patient with a long-term supply of CAR T cells. NK cells harboring the CAR do not require thymic maturation and are recruited to the tumor and induce an immediate immune response. Likewise, myeloid progeny that express the CAR may also contribute to the anti-tumor response.

Treatment of cancer patients with a combination of HSCs modified with a lentiviral vector encoding a CAR and CAR T cells will be superior to a single CAR T cell infusion or single HSC CAR modified infusion.

This treatment approach is modeled in mice. Briefly, immunocompetent Balb/c mice are treated with busulfan to condition the mice for bone marrow transplant. The mice are implanted subcutaneously in the flank with A20 lymphoma cells and then receive an intravenous injection of control Balb/c HSCs or Balb/c HSCs modified with a lentiviral vector encoding an anti-mouse CD19 CAR. When the A20 tumor reaches ˜100 mm³ in 7-9 days, subgroups of each of the two cohorts are treated with vehicle, untransduced Balb/c T cells or Balb/c T cells modified with a lentiviral vector encoding an anti-mouse CD19 CAR. The two mouse subcohorts receiving the anti-mouse CD19 CAR T cells will clear the A20 tumors and endogenous B cells, but the other groups will not and will be removed from the experiment. Anti-mouse CD19 CAR T cells, when provided as a single treatment, have a limited lifespan in vivo and endogenous B cells return to pre-treatment levels by Day 81 post-treatment. The remaining two groups of mice are rechallenged with a subcutaneous injection of A20 cells on Day 80. The animals treated with the WT HSCs+anti-mouse CD19 CAR T cells will no longer have the latter in circulation and will develop progressively growing A20 tumors. In contrast, the animals treated with the Balb/c HSCs modified with a lentiviral vector encoding an anti-mouse CD19 CAR will be continuously producing fresh anti-mouse CD19 CAR T cells and therefore will be resistant to A20 challenge and permanently depleted of B cells.

This treatment approach is also modeled in a nonhuman primate. Briefly, peripheral blood cells are collected from cynomolgus macaques, and T cells are isolated and modified with a lentiviral vector encoding an anti-cyno CD20 CAR and frozen for later use. B cells are isolated from the flow-through and frozen for later use. Subsequently, the same animal is treated with G-CSF (Amgen) at 50 μg/kg for three days and HSCs modified with a lentiviral vector encoding an anti-cyno CD20 CAR and frozen for later use. Following recovery for several weeks, animals receive pre-transplant conditioning as previously described (Colonna, Nature Comm, 2018) including a myeloablative dose of 500 to 550 cGy daily total body irradiation (TBI) delivered during two days (Ageyama, Comp Med, 2002) via a Varian Clinac 23EX Energy Linear Accelerator (Varian). Transplant recipients receive a central venous catheter surgically placed on the day of transplant which facilitates the administration of antiviral (acyclovir, 5-10 mg/kg IV daily; cidofovir, 3-5 mg/kg IV weekly) and antibacterial (vancomycin, 5-10 mg/kg daily; ceftazidime, 150 mg/kg IV daily; fluconazole, 5 mg/kg orally or IV daily) prophylaxis. On the day following TBI, animals are administered 1×10⁷ anti-cyno CD20 CAR T cells/kg+1×10⁷ anti-cyno CD20 HSC-CAR cells/kg. Whole blood support (irradiated whole blood or platelet-rich plasma) is administered as needed (i.e., when platelets count decreases below 50×10³ per μL, or hemoglobin drops below 9 g/dL, or significant hemorrhage is noted). Following administration, the anti-cyno CD20 CAR T cells administered at the time of transplantation clear any residual B cells present in the animals. As the anti-cyno CD20 CAR T cells terminally differentiate, they are eliminated by 40 days post-transplant, resulting in the re-appearance of B cells in the peripheral blood. However, in this experiment, fresh anti-cyno CD20 CAR T cells differentiated from the anti-cyno CD20 HSC-CAR cells populate the animal, permanently depleting B cells from the peripheral blood and lymphoid tissues. Anti-cyno CD20 HSC-CAR cells engraftment is confirmed in the animal: a) presence of CD34+anti-cyno CD20 CAR+ cells in the bone marrow 60 days after transplantation is demonstrated by in situ hybridization and flow cytometry, b) presence of mature anti-cyno CD20 CAR T cells in the peripheral blood of the animal 60 days after transplant is demonstrated (beyond the time terminally differentiated CAR T cells from the original transplant would survive), c) permanent depletion of B cells from peripheral blood, bone marrow and tissues of the recipient animal is demonstrated; and d) when challenged with autologous B cells via IV injection on Day 60, expansion of anti-cyno CD20 CAR T cells and depletion of recurrent B cells is demonstrated.

In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A method of treating a cancer in a subject comprising:
(a) administering to the subject, an effective amount of human CD34⁺ hematopoietic stem and progenitor cells transduced with a lentiviral vector encoding a first engineered antigen receptor, wherein the first engineered antigen receptor comprises a binding domain that binds one or more target antigens present on a cancer cell; and
(b) administering to the subject, an effective amount of human immune effector cells transduced with a lentiviral vector encoding a second engineered antigen receptor;
thereby treating the cancer in the subject.

2. The method of claim 1, wherein the human CD34⁺ hematopoietic stem and progenitor cells are allogenic to the subject.

3. The method of claim 1, wherein the human CD34⁺ hematopoietic stem and progenitor cells are autologous to the subject.

4. The method of any one of claims 1 to 3, wherein the human immune effector cells are allogenic to the subject.

5. The method of any one of claims 1 to 3, wherein the human immune effector cells are autologous to the subject.

6. The method of any one of claims 1 to 5, wherein the human immune effector cells comprise T cells.

7. The method of any one of claims 1 to 6, wherein the human immune effector cells comprise T cells that express CD3⁺, CD4⁺, CD8⁺, or a combination thereof.

8. The method of any one of claims 1 to 7, wherein the human immune effector cells comprise cytotoxic T lymphocytes (CTLs), tumor infiltrating lymphocytes (TILs), and/or helper T cells.

9. The method of any one of claims 1 to 5, wherein the human immune effector cells comprise natural killer (NK) cells or natural killer T (NKT) cells.

10. The method of any one of claims 1 to 9, wherein the cancer is a solid cancer.

11. The method of any one of claims 1 to 10, wherein the cancer is a solid cancer selected from the group consisting of: adrenal cancer, adrenocortical carcinoma, anal cancer, appendix cancer, astrocytoma, atypical teratoid/rhabdoid tumor, basal cell carcinoma, bile duct cancer, bladder cancer, bone cancer, brain/CNS cancer, breast cancer, bronchial tumors, cardiac tumors, cervical cancer, cholangiocarcinoma, chondrosarcoma, chordoma, colon cancer, colorectal cancer, craniopharyngioma, ductal carcinoma in situ (DCIS) endometrial cancer, ependymoma, esophageal cancer, esthesioneuroblastoma, Ewing's sarcoma, extracranial germ cell tumor, extragonadal germ cell tumor, eye cancer, fallopian tube cancer, fibrous histiosarcoma, fibrosarcoma, gallbladder cancer, gastric cancer, gastrointestinal carcinoid tumors, gastrointestinal stromal tumor (GIST), germ cell tumors, glioma, glioblastoma, head and neck cancer, hemangioblastoma, hepatocellular cancer, hypopharyngeal cancer, intraocular melanoma, kaposi sarcoma, kidney cancer, laryngeal cancer, leiomyosarcoma, lip cancer, liposarcoma, liver cancer, lung cancer, non-small cell lung cancer, lung carcinoid tumor, malignant mesothelioma, medullary carcinoma, medulloblastoma, menangioma, melanoma, Merkel cell carcinoma, midline tract carcinoma, mouth cancer, myxosarcoma, myelodysplastic syndrome, myeloproliferative neoplasms, nasal cavity and paranasal sinus cancer, nasopharyngeal cancer, neuroblastoma, oligodendroglioma, oral cancer, oral cavity cancer, oropharyngeal cancer, osteosarcoma, ovarian cancer, pancreatic cancer, pancreatic islet cell tumors, papillary carcinoma, paraganglioma, parathyroid cancer, penile cancer, pharyngeal cancer, pheochromocytoma, pinealoma, pituitary tumor, pleuropulmonary blastoma, primary peritoneal cancer, prostate cancer, rectal cancer, retinoblastoma, renal cell carcinoma, renal pelvis and ureter cancer, rhabdomyosarcoma, salivary gland cancer, sebaceous gland carcinoma, skin cancer, soft tissue sarcoma, squamous cell carcinoma, small cell lung cancer, small intestine cancer, stomach cancer, sweat gland carcinoma, synovioma, testicular cancer, throat cancer, thymus cancer, thyroid cancer, urethral cancer, uterine cancer, uterine sarcoma, vaginal cancer, vascular cancer, vulvar cancer, and Wilms Tumor.

12. The method of any one of claims 1 to 11, wherein the cancer is a solid cancer selected from the group consisting of: liver cancer, pancreatic cancer, lung cancer, breast cancer, bladder cancer, brain cancer, bone cancer, thyroid cancer, kidney cancer, and skin cancer.

13. The method of any one of claims 1 to 9, wherein the cancer is a liquid cancer or hematological cancer.

14. The method of claim 13, wherein the hematological cancer is a B cell malignancy.

15. The method of claim 14, wherein the B cell malignancy is selected from the group consisting of: leukemias, lymphomas, and myelomas.

16. The method of claim 14 or claim 15, wherein the B cell malignancy is selected from the group consisting of: acute lymphocytic leukemia (ALL), acute myeloid leukemia (AML), myeloblastic, promyelocytic, myelomonocytic, monocytic, erythroleukemia, hairy cell leukemia (HCL), chronic lymphocytic leukemia (CLL), and chronic myeloid leukemia (CML), chronic myelomonocytic leukemia (CMML) and polycythemia vera, Hodgkin lymphoma, nodular lymphocyte-predominant Hodgkin lymphoma, Burkitt lymphoma, small lymphocytic lymphoma (SLL), diffuse large B-cell lymphoma, follicular lymphoma, immunoblastic large cell lymphoma, precursor B-lymphoblastic lymphoma, mantle cell lymphoma, marginal zone lymphoma, mycosis fungoides, anaplastic large cell lymphoma, Sézary syndrome, precursor T-lymphoblastic lymphoma, multiple myeloma, overt multiple myeloma, smoldering multiple myeloma, plasma cell leukemia, non-secretory myeloma, IgD myeloma, osteosclerotic myeloma, solitary plasmacytoma of bone, and extramedullary plasmacytoma.

17. The method of any one of claims 14 to 16, wherein the B cell malignancy is multiple myeloma.

18. The method of any one of claims 1 to 17, wherein the one or more target antigens is selected from the group consisting of: tumor associated antigens (TAA), tumor specific antigens (TSA), NKG2D ligands, γδ T cell receptor (TCR) ligands, and αβ TCR ligands.

19. The method of any one of claims 1 to 18, wherein the one or more target antigens is selected from the group consisting of: alpha folate receptor (FRα), αvβ6 integrin, B cell maturation antigen (BCMA), B7-H3 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IL-10Rα, IL-13Rα2, Kappa, cancer/testis antigen 2 (LAGE-1A), Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), trophoblast glycoprotein (TPBG), UL16-binding protein (ULBP) 1, ULBP2, ULBP3, ULBP4, ULBP5, ULBP6, vascular endothelial growth factor receptor 2 (VEGFR2), and Wilms tumor 1 (WT-1).

20. The method of any one of claims 1 to 19, wherein the one or more target antigens is selected from the group consisting of: BCMA, CD19, CD20, CD22, CD23, CD33, CD37, CD38, CD52, CD79a, CD79b, CD80, C123, and HLA-DR.

21. The method of any one of claims 1 to 20, wherein the one or more target antigens comprises BCMA.

22. The method of any one of claims 1 to 20, wherein the one or more target antigens comprises CD19.

23. The method of any one of claims 1 to 20, wherein the one or more target antigens comprises CD20.

24. The method of any one of claims 1 to 20, wherein the one or more target antigens comprises CD22.

25. The method of any one of claims 1 to 20, wherein the one or more target antigens comprises CD23.

26. The method of any one of claims 1 to 20, wherein the one or more target antigens comprises CD33.

27. The method of any one of claims 1 to 20, wherein the one or more target antigens comprises CD37.

28. The method of any one of claims 1 to 20, wherein the one or more target antigens comprises CD38.

29. The method of any one of claims 1 to 20, wherein the one or more target antigens comprises CD79a.

30. The method of any one of claims 1 to 20, wherein the one or more target antigens comprises CD79b.

31. The method of any one of claims 1 to 20, wherein the one or more target antigens comprises CD123.

32. The method of any one of claims 1 to 31, wherein the cancer expresses a first target antigen and a second target antigen.

33. The method of claim 32, wherein the first and second target antigens are expressed on different cancer cells.

34. The method of claim 32, wherein the first and second target antigens are expressed on the same cancer cells.

35. The method of any one of claims 1 to 34, wherein the first and second engineered antigen receptors are selected from the group consisting of: a chimeric antigen receptor (CAR), an αβ T cell receptor (αβ-TCR), a γδ T cell receptor (γδ-TCR), and a dimerizing agent regulated immunoreceptor complex (DARIC).

36. The method of any one of claims 1 to 35, wherein the first and second engineered antigen receptors are the same.

37. The method of any one of claims 1 to 36, wherein the first and second engineered antigen receptors are a CAR.

38. The method of claim 37, wherein the CAR comprises:
(a) one or more target antigen binding domains;
(b) a transmembrane domain;
(c) one or more intracellular costimulatory signaling domains; and/or
(d) a primary signaling domain.

39. The method of claim 38, wherein the one or more target antigen binding domains is selected from the group consisting of: a Camel Ig, a Llama Ig, an Alpaca Ig, Ig NAR, a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), and a single-domain antibody (sdAb, a camelid VHH, Nanobody).

40. The method of claim 38 or claim 39, wherein the one or more target antigen binding domains comprises one or more scFvs.

41. The method of claim 38 or claim 39, wherein the one or more target antigen binding domains comprises one or more VHHs.

42. The method of any one of claims 38 to 41, wherein the CAR comprises:
(a) an scFv;
(b) a CD28 transmembrane domain or a CD8α transmembrane domain;
(c) a 4-1BB, OX-40, or CD28 costimulatory domain; and
(d) a CD3ζ primary signaling domain.

43. The method of any one of claims 38 to 41, wherein the CAR comprises:
(a) a VHH;
(b) a CD28 transmembrane domain or a CD8α transmembrane domain;
(c) a 4-1BB, OX-40, or CD28 costimulatory domain; and
(d) a CD3ζ primary signaling domain.

44. The method of any one of claims 1 to 36, wherein the first and second engineered antigen receptors are a αβ-TCR.

45. The method of any one of claims 1 to 36, wherein the first and second engineered antigen receptors are a DARIC.

46. A method of treating a B cell malignancy in a subject comprising:
(a) administering to the subject, an effective amount of human CD34⁺ hematopoietic stem and progenitor cells transduced with a lentiviral vector encoding a CAR, wherein the CAR comprises a binding domain that binds one or more target antigens present on a malignant B cell; and
(b) administering to the subject, an effective amount of human immune effector cells transduced with the lentiviral vector encoding the CAR;
thereby treating the B cell malignancy in the subject.

47. The method of claim 46, wherein the human CD34⁺ hematopoietic stem and progenitor cells are allogenic to the subject.

48. The method of claim 46, wherein the human CD34⁺ hematopoietic stem and progenitor cells are autologous to the subject.

49. The method of any one of claims 46 to 48, wherein the human immune effector cells are allogenic to the subject.

50. The method of any one of claims 46 to 48, wherein the human immune effector cells are autologous to the subject.

51. The method of any one of claims 46 to 50, wherein the human immune effector cells comprise T cells.

52. The method of any one of claims 46 to 51, wherein the human immune effector cells comprise T cells that express CD3⁺, CD4⁺, CD8⁺, or a combination thereof.

53. The method of any one of claims 46 to 52, wherein the human immune effector cells comprise cytotoxic T lymphocytes (CTLs), tumor infiltrating lymphocytes (TILs), and/or helper T cells.

54. The method of any one of claims 46 to 50, wherein the human immune effector cells comprise natural killer (NK) cells or natural killer T (NKT) cells.

55. The method of any one of claims 46 to 54, wherein the B cell malignancy is selected from the group consisting of: leukemias, lymphomas, and myelomas.

56. The method of any one of claims 46 to 55, wherein the B cell malignancy is selected from the group consisting of: acute lymphocytic leukemia (ALL), acute myeloid leukemia (AML), myeloblastic, promyelocytic, myelomonocytic, monocytic, erythroleukemia, hairy cell leukemia (HCL), chronic lymphocytic leukemia (CLL), and chronic myeloid leukemia (CML), chronic myelomonocytic leukemia (CMML) and polycythemia vera, Hodgkin lymphoma, nodular lymphocyte-predominant Hodgkin lymphoma, Burkitt lymphoma, small lymphocytic lymphoma (SLL), diffuse large B-cell lymphoma, follicular lymphoma, immunoblastic large cell lymphoma, precursor B-lymphoblastic lymphoma, mantle cell lymphoma, marginal zone lymphoma, mycosis fungoides, anaplastic large cell lymphoma, Sézary syndrome, precursor T-lymphoblastic lymphoma, multiple myeloma, overt multiple myeloma, smoldering multiple myeloma, plasma cell leukemia, non-secretory myeloma, IgD myeloma, osteosclerotic myeloma, solitary plasmacytoma of bone, and extramedullary plasmacytoma.

57. The method of any one of claims 46 to 56, wherein the B cell malignancy is multiple myeloma.

58. The method of any one of claims 46 to 57, wherein the one or more target antigens is selected from the group consisting of: BCMA, CD19, CD20, CD22, CD23, CD33, CD37, CD38, CD52, CD79a, CD79b, CD80, and CD123.

59. The method of any one of claims 46 to 58, wherein the one or more target antigens comprises BCMA.

60. The method of any one of claims 46 to 58, wherein the one or more target antigens comprises CD19.

61. The method of any one of claims 46 to 58, wherein the one or more target antigens comprises CD20.

62. The method of any one of claims 46 to 58, wherein the one or more target antigens comprises CD22.

63. The method of any one of claims 46 to 58, wherein the one or more target antigens comprises CD23.

64. The method of any one of claims 46 to 58, wherein the one or more target antigens comprises CD33.

65. The method of any one of claims 46 to 58, wherein the one or more target antigens comprises CD37.

66. The method of any one of claims 46 to 58, wherein the one or more target antigens comprises CD38.

67. The method of any one of claims 46 to 58, wherein the one or more target antigens comprises CD79a.

68. The method of any one of claims 46 to 58, wherein the one or more target antigens comprises CD79b.

69. The method of any one of claims 46 to 58, wherein the one or more target antigens comprises CD123.

70. The method of any one of claims 46 to 69, wherein the B cell malignancy expresses a first target antigen and a second target antigen.

71. The method of claim 70, wherein the first and second target antigens are expressed on different malignant B cells.

72. The method of claim 70, wherein the first and second target antigens are expressed on the same malignant B cells.

73. The method of any one of claims 46 to 72, wherein the CAR comprises:
(a) one or more target antigen binding domains;
(b) a transmembrane domain;
(c) one or more intracellular costimulatory signaling domains; and/or
(d) a primary signaling domain.

74. The method of claim 73, wherein the one or more target antigen binding domains is selected from the group consisting of: a Camel Ig, a Llama Ig, an Alpaca Ig, Ig NAR, a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), and a single-domain antibody (sdAb, a camelid VHH, Nanobody).

75. The method of claim 73 or claim 74, wherein the one or more target antigen binding domains comprises one or more scFvs.

76. The method of claim 73 or claim 74, wherein the one or more target antigen binding domains comprises one or more VHHs.

77. The method of any one of claims 46 to 76, wherein the CAR comprises:
(a) an scFv;
(b) a CD28 transmembrane domain or a CD8α transmembrane domain;
(c) a 4-1BB, OX-40, or CD28 costimulatory domain; and
(d) a CD3ζ primary signaling domain.

78. The method of any one of claims 46 to 76, wherein the CAR comprises:
(a) a VHH;
(b) a CD28 transmembrane domain or a CD8α transmembrane domain;
(c) a 4-1BB, OX-40, or CD28 costimulatory domain; and
(d) a CD3ζ primary signaling domain.

79. A method of treating a leukemia, lymphoma, or myeloma in a subject comprising:
(a) administering to the subject, an effective amount of autologous human CD34⁺ hematopoietic stem and progenitor cells transduced with a lentiviral vector encoding a CAR, wherein the CAR comprises an scFv or VHH that binds a target antigen; a CD28 transmembrane domain or a CD8α transmembrane domain; a 4-1BB, OX-40, or CD28 costimulatory domain; and a CD3ζ primary signaling domain; and
(b) administering to the subject, an effective amount of autologous human immune effector cells transduced with the lentiviral vector encoding the CAR;
thereby treating the leukemia, lymphoma, or myeloma in the subject.

80. A method of treating a leukemia, lymphoma, or myeloma in a subject comprising:
(a) administering to the subject, an effective amount of allogenic human CD34⁺ hematopoietic stem and progenitor cells transduced with a lentiviral vector encoding a CAR, wherein the CAR comprises an scFv or VHH that binds a target antigen; a CD28 transmembrane domain or a CD8α transmembrane domain; a 4-1BB, OX-40, or CD28 costimulatory domain; and a CD3ζ primary signaling domain; and
(b) administering to the subject, an effective amount of allogenic human immune effector cells transduced with the lentiviral vector encoding the CAR;
thereby treating the leukemia, lymphoma, or myeloma in the subject.

81. The method of claim 79 or claim 80, wherein the human immune effector cells comprise T cells.

82. The method of any one of claims 79 to 81, wherein the human immune effector cells comprise T cells that express CD3⁺, CD4⁺, CD8⁺, or a combination thereof.

83. The method of any one of claims 79 to 82, wherein the human immune effector cells comprise cytotoxic T lymphocytes (CTLs), tumor infiltrating lymphocytes (TILs), and/or helper T cells.

84. The method of claim 79 or claim 80, wherein the human immune effector cells comprise natural killer (NK) cells or natural killer T (NKT) cells.

85. The method of any one of claims 79 to 84, wherein the target antigen is selected from the group consisting of: BCMA, CD19, CD20, CD22, CD23, CD33, CD37, CD38, CD52, CD79a, CD79b, CD80, and CD123.

86. The method of any one of claims 46 to 58, wherein the target antigen is BCMA, CD19, CD20, CD22, CD33, CD79a, CD79b, CD80, or CD123.

87. A method of preparing a combination adoptive cell therapy product for treating cancer comprising:
(a) transducing a population of cells comprising human CD34⁺ hematopoietic stem and progenitor cells with a lentiviral vector encoding a first engineered antigen receptor that binds one or more antigens present on a cancer cell;
(b) transducing a population of cells comprising human immune effector cells with a lentiviral vector encoding a second engineered antigen receptor that binds one or more antigens present on a malignant B cell; and
(c) formulating the populations of cells for administration to a subject that has, or that has been diagnosed, with a cancer.

88. The method of claim 87, wherein the human CD34⁺ hematopoietic stem and progenitor cells are allogenic to the subject.

89. The method of claim 87, wherein the human CD34⁺ hematopoietic stem and progenitor cells are autologous to the subject.

90. The method of any one of claims 87 to 89, wherein the human immune effector cells are allogenic to the subject.

91. The method of any one of claims 87 to 89, wherein the human immune effector cells are autologous to the subject.

92. The method of any one of claims 87 to 91, wherein the human immune effector cells comprise T cells, NK cells, or NKT cells.

93. The method of any one of claims 87 to 92, wherein the first and second engineered antigen receptors are selected from the group consisting of: a chimeric antigen receptor (CAR), an αβ T cell receptor (αβ-TCR), a γδ T cell receptor (γδ-TCR), and a dimerizing agent regulated immunoreceptor complex (DARIC).

94. The method of any one of claims 87 to 93, wherein the first and second engineered antigen receptors are the same.

95. The method of any one of claims 87 to 94, wherein the first and second engineered antigen receptors are a CAR.

96. The method of any one of claims 87 to 94, wherein the first and second engineered antigen receptors are a αβ-TCR.

97. The method of any one of claims 87 to 94, wherein the first and second engineered antigen receptors are a DARIC.
